(19) United States
(12) Patent Application Publication
(10) Pub. No.: US 2026/0108621 A1
(43) Pub. Date: Apr. 23, 2026

(54) Immune Cell Conjugates and Methods for Producing and Using the Same

(71) Applicant: Sandigene Inc., San Diego, CA (US)

(72) Inventors: Yanwen FU, San Diego, CA (US); Reyna Koreen V. Lim, San Diego, CA (US); Changyou Lin, San Diego, CA (US); Moli Liu, San Diego, CA (US)

(73) Assignee: Sandigene Inc., San Diego, CA (US)

(21) Appl. No.: 19/360,236

(22) Filed: Oct. 16, 2025

Related U.S. Application Data
(60) Provisional application No. 63/708,697, filed on Oct. 17, 2024.

(51) Int. Cl. A61K 47/69; A61P 35/00; C12N 5/00

(52) U.S. Cl. CPC A61K 47/6901; A61P 35/00; C12N 5/0006

(57) ABSTRACT

The present disclosure provides a surface modified immune cell comprising (i) a linker that is covalentiy bound to an immune cell surface and (ii) a non-antibody ligand covalently bound to said linker, wherein said ligand is selective to a receptor that is present in a target cell. The present disclosure also provides a method for producing and using the same.

### CROSS-REFERENCE TO RELATED APPLICATIONS

This application claims the priority benefit of U.S. Provisional Application No. 63/708,697, filed Oct. 17, 2024, which is incorporated herein by reference in its entirety.

### FIELD

The present disclosure relates to modifying immune cell surface to attach a non-antibody targeting conjugate and uses thereof.

### BACKGROUND

Chimeric antigen receptor T (CAR-T) cell therapies have grown rapidly recently in treating cancer and attempts to treat other immune-based clinical conditions, such as organ transplantation and rheumatologic diseases like lupus. In general, CAR-T based cancer treatments have been focused on non-solid cancers, such as acute lymphoblastic leukemia, diffuse large B-cell lymphoma, as well as targeting other blood cancer antigens, including CD30 in refractory Hodgkin's lymphoma; targeting CD33, CD123, and FLT3 in acute myeloid leukemia (AML); and BCMA in multiple myeloma. While a first CAR-macrophages (CAR-M) have entered the clinic trial for the treatment of solid tumors, in general solid tumors have presented a more difficult target as CAR-T cells do not efficiently enter into the center of solid tumor masses. Furthermore, the hostile tumor microenvironment presented by the solid tumors suppresses T cell activity.

Immune effector cells such as lymphocytes, macrophages, dendritic cells, natural killer cells, and cytotoxic T lymphocytes work together to defend the body against cancer by targeting abnormal antigens expressed on the surface of tumor cells. Most conventional CAR-T therapies for treating cancer involve using an antibody that is selective for cancer cells. In general, these antibodies are attached to immune effector cells to seek out and target cancer cells selectively as schematically illustrated in FIG. 1.

Unfortunately, some of the limitations of conventional immunotherapies include, but are not limited to, requiring modification of autologous immune cells, production of cancer cell selective antibodies. Such requirements render conventional cancer immunotherapies time consuming, laborious, and in many cases have limited applications in solid tumors.

Accordingly, there is a need for alternative cellular therapy to overcome some of the problems of conventional chimeric antigen receptor therapy.

### SUMMARY

Some aspects of the disclosure are based on the discovery by the present inventor that one can take advantage of certain target cells having a significantly higher amount of receptors compared to non-target cells. This difference in the amount of receptors in the target cell allows one to utilize surface modified immune cells having a ligand that can selectively and effectively bind to the target cell due at least in part to higher amount of receptors present on the target (e.g., cancer) cell.

Accordingly, one particular aspect of the disclosure provides a surface modified immune cell comprising (i) a linker that is covalently bound to an immune cell surface and (ii) a ligand covalently bound to said linker, wherein said ligand is selective to a receptor that is present in a target cell. In some embodiments, the immune cell is an innate immune cell. Still in other embodiments, the immune cell is a T cell, B cell, natural killer (NK) cell, macrophage, neutrophil, dendritic cell, mast cell, eosinophil, or basophil. In one particular embodiment, the immune cell is γδT cell, i.e., gdT cell.

Still in other embodiments, the linker comprises polyethylene glycol (PEG), polyvinyl acid (PVA), polycaprolactone (PCL), polylactic acid (PLA), poly(lactic-co-glycolic acid) (PLGA), polyurethane (PU), polyglycolic acid (PGA), polyethylene oxide (PEO), polypeptide, aliphatic chain, or a combination thereof. In one particular embodiment, the linker comprises from about two to about ten monomeric ethylene glycol units. In further embodiments, the immune cell comprises a plurality of said linker.

Yet in other embodiments, the ligand comprises folate, folate analogs, such as 5-methyltetrahydrofolate, methotrexate, pralatrexate, raltitrexed and pemetrexed, or a mixture thereof. Still in other embodiments, the ligand is capable of binding to a B cell, plasma cell, T cell, monocyte, or macrophage that has a pathogenic role in human autoimmune diseases.

Another aspect of the disclosure provides a method for treating cancer in a subject, said method comprising administering a composition comprising a surface modified immune cell disclosed herein.

In some embodiments, the cancer is a solid cancer. Still in other embodiments, the cancer comprises ovarian cancer, breast cancer, lung cancer, stomach cancer, colorectal cancer, brain cancer, melanoma, sarcoma, lymphoma, blastoma, carcinosarcoma, carcinoma, kidney cancer, bladder cancer, pancreatic cancer, cervical cancer, or uterine cancer.

Still in other embodiments, the cancer is a blood cancer. Yet in other embodiments, the cancer comprises multiple myeloma, hematopoietic malignancies of myeloid origin, such as myelogenous leukemias.

In further embodiments, the immune cell is an innate immune cell. Still in other embodiments, said immune cell is an allogeneic immune cell. In one particular embodiments, the composition comprises a plurality of allogeneic immune cells.

Further aspect of the disclosure provides a method for producing a surface modified immune cell. The method includes:
(a) contacting an immune cell with a linker having a proximal end and a distal end under conditions sufficient to form a covalent bond between said immune cell surface and said proximal end of said linker; and
(b) contacting said immune cell of step (a) with a ligand under conditions sufficient to form a covalent bond between said distal end of said linker and said ligand to produce said surface modified immune cell, wherein said ligand is capable of binding to a receptor of a target cell; or

Alternatively, the method includes contacting an immune cell with a linker having a proximal end and a distal end under conditions sufficient to form a covalent bond between said immune cell surface and said proximal end of said linker to produce said surface modified immune cell, wherein said distal end of said linker comprises a covalently bonded ligand that is capable of binding to a receptor of a target cell.

In some embodiments, said immune cell is obtained from peripheral blood mononuclear cells from a plurality of donors.

Still in other embodiments, said immune cell is obtained from peripheral blood mononuclear cells from a single donor.

### BRIEF DESCRIPTION OF THE DRAWINGS

FIG. 1 is a schematic illustration of a cellular immunotherapy for treating cancer using a CAR-T cell.

FIG. 2 is a schematic illustration of one particular embodiment of a method of the disclosure for producing a surface modified immune cell.

FIG. 3 is a schematic illustration of one particular method of producing surface modified immune cells from allogeneic donor(s).

FIG. 4 shows one method for expanding immune cells from PBMC.

FIG. 5 shows one embodiment for one-step cell surface modification of immune cells.

FIG. 6 illustrates one embodiment for a two-step cell surface modification of immune cells.

FIG. 7 shows examples of folate-linker compounds that can be used in one-step immune cell surface modification.

FIG. 8 shows some possible linkers and functional groups that can be used in two-step immune cell surface modification.

FIG. 9 shows other examples of possible linkers and functional groups that can be used in two-step immune cell surface modification.

FIG. 10 shows other examples of folate-linker compounds that can be used in two-step immune cell surface modification.

FIG. 11 shows still other examples of folate-linker compounds that can be used in two-step immune cell surface modification.

FIG. 12 is a flow cytometry of one possible method for attaching folates to gdT cell surface. As can be seen, the graph shows attaching folates to gdT cell surface using one method of the disclosure is highly efficient.

FIG. 13A shows folate-gdT conjugate cytotoxicity against THP-1 tumor cells at E:T (Effector cells:Tumor cells) ratio of 3:1 (from left to right: γδT cells only; folate-conjugated γδT cells; free folate+γδT cells; and control, i.e., tumor cells only).

FIG. 13B shows folate-gdT conjugate cytotoxicity against THP-1 tumor cells at E:T ratio of 1:1 (from left to right: γδT cells only; folate-conjugated γδT cells; and free folate+γδT cells)

FIG. 13C shows folate-gdT conjugate cytotoxicity against THP-1 tumor cells at E:T ratio of 1:3 (from left to right: γδT cells only; folate-conjugated γδT cells; and free folate+γδT cells)

FIG. 13D shows folate-gdT conjugate cytotoxicity against THP-1 tumor cells at ET ratio of 1:9 (from left to right: γδT cells only; folate-conjugated γδT cells; and free folate+γδT cells)

FIG. 14A shows folate-gdT conjugate cytotoxicity against KB tumor cells at E:T (Effector cells:Tumor cells) ratio of 3:1 (from left to right: γδT cells only; folate-conjugated γδT cells; free folate+γδT cells; and control, i.e., tumor cells only). an

FIG. 14B shows folate-gdT conjugate cytotoxicity against KB tumor cells at E:T ratio of 1:1 (from left to right: γδT cells only; folate-conjugated γδT cells; and free folate+γδT cells)

FIG. 14C shows folate-gdT conjugate cytotoxicity against KB tumor cells at E:T ratio of 1:3 (from left to right: γδT cells only; folate-conjugated γδT cells; and free folate+γδT cells)

FIG. 14D shows folate-gdT conjugate cytotoxicity against KB tumor cells at E:T ratio of 1:9 (from left to right: γδT cells only; folate-conjugated γδT cells; and free folate+γδT cells).

FIG. 15A shows folate-gdT conjugate cytotoxicity against HL tumor cells at E:T (Effector cells:Tumor cells) ratio of 3:1 (from left to right: γδT cells only; folate-conjugated γδT cells; free folate+γδT cells; and control, i.e., tumor cells only).

FIG. 15B shows folate-gdT conjugate cytotoxicity against HL tumor cells at E:T ratio of 1:1 (from left to right: γδT cells only; folate-conjugated γδT cells; and free folate+γδT cells)

FIG. 15C shows folate-gdT conjugate cytotoxicity against HL tumor cells at E:T ratio of 1:3 (from left to right: γδT cells only; folate-conjugated γδT cells; and free folate+γδT cells)

FIG. 15D shows folate-gdT conjugate cytotoxicity against HL tumor cells at E:T ratio of 1:9 (from left to right: γδT cells only; folate-conjugated γδT cells; and free folate+γδT cells).

FIG. 16A shows folate-gdT conjugate cytotoxicity against Toledo tumor cells at E:T (Effector cells:Tumor cells) ratio of 3:1 (from left to right: γδT cells only; folate-conjugated γδT cells; free folate+γδT cells; and control, i.e., tumor cells only).

FIG. 16B shows folate-gdT conjugate cytotoxicity against Toledo tumor cells at E:T ratio of 1:1 (from left to right: γδT cells only; folate-conjugated γδT cells; and free folate+γδT cells)

FIG. 16C shows folate-gdT conjugate cytotoxicity against Toledo tumor cells at E:T ratio of 1:3 (from left to right: γδT cells only; folate-conjugated γδT cells; and free folate+γδT cells)

FIG. 16D shows folate-gdT conjugate cytotoxicity against Toledo tumor cells at E:T ratio of 1:9 (from left to right: γδT cells only; folate-conjugated γδT cells; and free folate+γδT cells).

FIG. 17 shows a summary of folate-gdT conjugate cytotoxicity against THP-1 tumor cells based on FIGS. 13A-13D.

FIG. 18 shows a summary of folate-gdT conjugate cytotoxicity against KB tumor cells based on FIGS. 14A-14D.

FIG. 19 shows a summary of folate-gdT conjugate cytotoxicity against HL-60 tumor cells based on FIGS. 15A-15D.

FIG. 20 shows a summary of folate-gdT conjugate cytotoxicity against Toledo tumor cells based on FIGS. 16A-16D.

FIG. 21 is a graph showing cytotoxicity of folate conjugated γδT cells against tumor cells at various ratio of E:T.

FIG. 22 is a graph showing binding of folate conjugated on γδT cell surface and human folate receptors.

FIG. 23 is a graph showing results of Granzyme B upregulation in Folate-Conjugated γδT cells following tumor cell-mediated activation.

FIG. 24 is a graph showing results of cytotoxity test of folate conjugated gdT cells against peripheral blood mononuclear cells (PBMCs). (PBMC as control; gdT=γδT cells).

FIG. 25 is IVIS images showing in vivo efficacy results of treating THP-1 human leukemia xenograft model with surface modified immune cells of the disclosure.

FIG. 26 is a graphic representation of results of FIG. 25 showing comparison of overall survival rate of THP-1 human leukemia xenograft models treated with surface modified immune cells of the disclosure compared to control or untreated models.

FIG. 27 is a graph of the total bioluminescence imaging (BLI) signal from FIG. 25 as quantitative measure of tumor burden.

FIG. 28 is a graph of body weight of THP-1 human leukemia xenograft models of FIG. 25 during in vivo treatment.

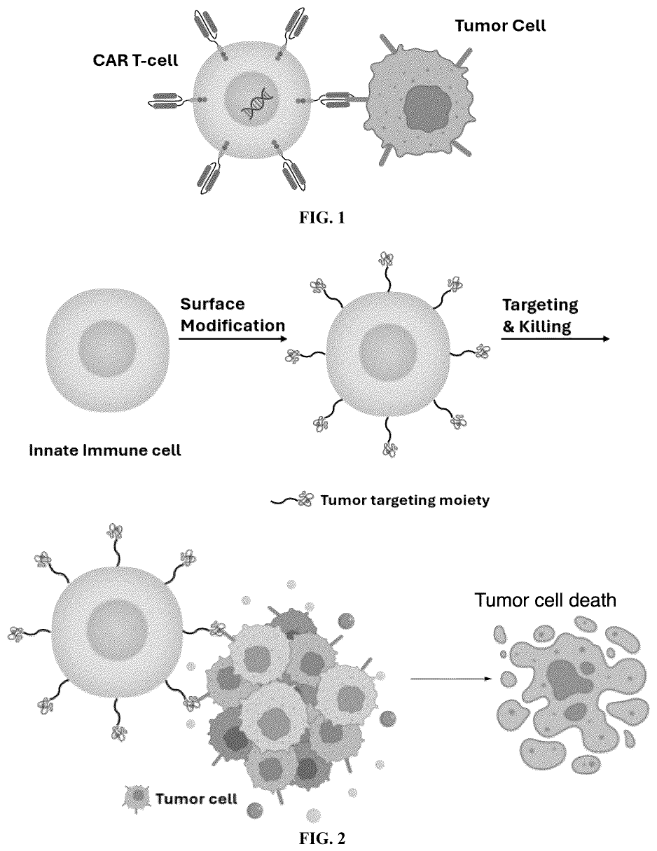

### DETAILED DESCRIPTION

The terms “comprise(s),” “include(s),” “having,” “has,” “can,” “contain(s),” and variants thereof, as used herein, are intended to be open-ended transitional phrases, terms, or words that do not preclude the possibility of additional steps or components or ingredients. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not. Accordingly, the transitional phrases “consisting of” and “consisting essentially of” may be interpreted to be subsets of the open-ended transitional phrases, such as “comprising” and “including,” such that any use of an open-ended phrase to introduce a recitation of a series of elements, limitations, components, ingredients, materials, or steps should be interpreted to also disclose recitation of the series of elements, limitations, components, ingredients, materials, or steps using the closed terms “consisting of” and “consisting essentially of.” For example, the recitation of a composition “comprising” components A, B and C should be interpreted as also disclosing a composition “consisting of” components A, B, and C as well as a composition “consisting essentially of” components A, B, and C.

Chimeric antigen receptors (CARs)—also known as chimeric immunoreceptors, chimeric T cell receptors or artificial T cell receptors—are receptor proteins that have been engineered to give T cells the ability to target a specific antigen. While there is much potential for CAR-T cell therapies, a number of factors limit the widespread development and efficacy of conventional CAR-T cell therapies. For example, most conventional CAR-T cell therapies utilize autologous T-cells requiring extended manufacturing time, high cost, and a limited period in which these therapies may be genetically modified to enhance their efficacy.

Some aspects of the disclosure avoid one or more of these shortcomings of conventional chimeric antigen receptor-based therapies. In some embodiments, methods and compositions of the disclosure utilize a surface modified immune cell comprising a ligand that is capable of selectively binding to a receptor that is present in a target cell. Unlike conventional chimeric antigen receptor-based therapies, the ligand used in the present disclosure does not comprise an antibody. Unless the context requires otherwise, the term “ligand” refers to any substance that is capable of binding selectively with a receptor. However, it should be appreciated that the term ligand does not include an antibody. A ligand can be an oligonucleotide, e.g., an aptamer or a short inhibitory nucleotide (i.e., about 200 nucleotides or less, typically about 150 nucleotides or less, often about 100 nucleotides or less, more often about 75 nucleotides or less, and most often about 50 nucleotides or less), an oligopeptide (including proteins, hormone, etc.), an enzyme, a substrate, a drug, a receptor agonist, a partial agonist, a mixed agonist, an antagonist, a hormone, a vitamin, a coenzyme, a cofactor, a toxin, a regulatory factor, a carbohydrate, a molecular mimic, biotin, or a derivative of any one of these molecules as well as a non-oligonucleotide molecule, or any other molecule that is capable of selectively binding to a receptor that is present in the target cell. It should be appreciated that the receptor may be present in both the target and non-target cells (e.g., cancer and healthy cells). However, due to the abundance or increased amount of the receptors present in the target cells, the ligand can effectively bind selectively to the target cell. As used herein, the term “target cells” and any grammatical variations thereof refer to cells that causes clinical conditions or diseases, such as cancer, autoimmune disease, immunodeficiency disease, inflammatory disease, infection, cardiometabolic disease, senility, Alzheimer's disease, cognitive impairment, as well as any other diseases or clinical conditions caused by an “abnormal cells” or antigens known to one skilled in the art.

It should be appreciated that the terms “ligand” and “receptor” do not refer to any particular substance or size relationship. These terms are only operational terms that indicate selective binding between the ligand and the corresponding receptor where the moiety that is bound to a substrate surface is referred to as a receptor and any substance that selectively binds to the receptor is referred to as a ligand. Thus, if a substance is present in the target cell's surface then this substance is the receptor and the complementary or corresponding binding moiety that is present in the surface modified immune cell of the disclosure is the ligand.

In some embodiments, the ligand comprises folate, or a folate analog, such as 5-methyltetrahydrofolate, methotrexate, pralatrexate, raltitrexed, pemetrexed, or a mixture thereof. If the ligand is a chiral molecule, the ligand can be a racemic ligand, enantiomerically enriched ligand, (D)-isomer ligand, (L)-isomer ligand, etc. For example, ligand can be racemic folate, enantiomerically enriched folate, (D)-folate, (L)-folate, or any combination thereof. Other suitable ligands of the disclosure include, but are not limited to, aptamers that bind to folate receptors. The term “aptamer” is used herein to refer to an oligomer of artificial ssDNA, dsDNA, RNA, XNA, or peptide that recognizes and binds to a desired target molecule, e.g., folate receptor, by virtue of its shape or complementary nature of the nucleotides. See, e.g., PCT Publication Nos. WO92/14843, WO91/19813, and WO92/05285, the disclosures of which are incorporated by reference herein. Suitable aptamers can be readily prepared by one skilled in the art. For example, U.S. Pat. No. 5,637,459, issued on Jun. 10, 1997 to Burke et al., which is incorporated herein by reference in its entirety, discloses what is commonly known in the art as the SELEX process for preparing aptamers for a particular ligand. Aptamers that are suitable for methods of the disclosure can be produced by SELEX or other similar processes that are known to one skilled in the art. In some embodiments, the aptamer is typically 15 to 100 nucleobases in length. In other embodiments, the aptamer is 10 to 200 nucleobases long. It should be appreciated that selectivity of the aptamer for a particular ligand of interest can be readily determined by one skilled in the art during the aptamer production process. Other methods for producing a suitable aptamer include analyzing the structure or the amino acids sequence that make up the binding domain of a receptor, e.g., of FRα, and producing a complementary structured aptamer. Other methods for producing a suitable aptamer include using an artificial intelligence program such as, those available in https://alphafold.com/and https://www.ncbi.nlm.nih.gov/guide/howto/view-3d-struct-prot/as well as other online or internet websites as well as various commercial and open-source computer programs.

In general, the ligand is selected based on the receptor that is present in the desired target cells. The terms “desired target cell” and “target cell” including grammatical variations thereof, are used interchangeably herein and refer to cells to which the surface modified immune cells of the disclosure are designed to selectively bind. Similarly, the term “receptor” including grammatical variations thereof refers to a complementary moiety that is present on the target cell to which the ligand of the surface modified immune cell of the disclosure is designed or is capable of binding thereto. It should be appreciated that in some instances, non-target cells can also include a receptor. However, in some embodiments, the amount of receptors present in the target cells is greater than the amount of similar or same receptors present in non-target cells. In this manner, the effective concentration of the receptor is greater in target cells, thereby allowing a higher affinity of the ligand towards the target cell compared to a non-target cell. Accordingly, in some embodiments, ligand is selected such that the amount of corresponding receptors on the target cells is greater compare to the amount of receptors on the non-target cells.

As illustrated in FIG. 2, in some embodiments the ligand is selected such that the ligand selectively binds to a receptor that is present on the target cell surface. Accordingly, some embodiments of the disclosure provide a surface modified immune cell in which the ligand comprises folate or a folate analog. Folate Receptor alpha (FRα), also known as Folate Receptor 1 (FOLR1), is a cell surface glycosylphosphatidylinositol (GPI)-anchored glycoprotein. FRα has important functions relating to cell proliferation and survival. FRα mediates delivery of the physiological folate to the interior of cells. Interestingly, FRα is overexpressed in epithelial-derived tumors including ovarian, uterine, breast, endometrial, pancreatic, renal, lung, colorectal, and brain tumors. For example, FRα is overexpressed in (i) 14-74% of non-small-cell lung cancers; (ii) 72-100% of mesotheliomas; (iii) 20-50% of endometrial cancers; (iv) 35-68% of triple-negative breast cancers; and (v) 76-89% of epithelial ovarian cancers. It is believed that overexpression of FRα may render a growth advantage for cancer cells through mechanisms both relating to, as well as being independent of, folate uptake. Elevated FRα expression is also associated with lower disease-free interval (DFI) and poor overall survival (OS) in patients with disease of serous origin. Accordingly, some embodiments of the disclosure provide a ligand that selectively binds to FRα. By targeting FRα that are present and overexpressed in tumor cells allows treatment of cancer patient groups that do not adequately benefit from conventional cancer therapies.

In some embodiments, the ligand is capable of binding to an immune cell (e.g., B cell, plasma cell, T cell, monocyte, or macrophage) that has a pathogenic role in human autoimmune disease(s). Autoimmune diseases are caused by malfunctioning immune system, where immune cells mistakenly attack one's own healthy cells and tissues. This malfunction can be influenced by genetic factors, environmental triggers, infections, and other causes leading to chronic inflammation and damage to various organs. Thus, immune cells play central pathogenic roles in many human autoimmune diseases by recognizing self-antigens and triggering immune response to one's own healthy cells and/or tissues. Exemplary ligands that can be used to treat autoimmune diseases include, but are not limited to, a small molecule, peptide, or oligonucleotide that is capable of binding selectively with folate receptors, CD19, CD20, CD38, CD22 and/or BCMA, such as folate, 5-methyltetrahydrofolate, methotrexate, pralatrexate, raltitrexed, pemetrexed, or a combination thereof.

The surface modified immune cell of the disclosure can also include a linker that is covalently attached or bound to the immune cell surface. The length of the linker is sufficiently long enough to allow presentation of the ligand to the target cell without a significant steric interference from the immune cell. Typically, the length of the linker is at least about 6 chain atoms, typically at least about 8 atoms, often at least about 10 atoms, more often at least about 12 atoms, and most often at least about 13 atoms. As used herein, the term “length” when referring to a linker means the smallest number of atoms in a chain that directly links the immune cell surface to the ligand. For example, if a dimer of ethylene glycol (i.e., a moiety of the formula: —O—CH₂—CH₂—O—CH₂—CH₂—O—) is a linker, then the length of the linker is seven (7) atoms. When referring to a numerical value, the terms “about” and “approximately” are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean±20%, typically ±10%, often ±5% and more often ±1% of the numerical value. In general, however, where particular values are described in the application and claims, unless otherwise stated, the term “about” means within an acceptable error range for the particular value, typically within one standard deviation.

In some embodiments, the length of the linker ranges from about 13 atoms to about 44 atoms, typically from about 13 atoms to about 35 atoms, often from about 13 atoms to about 30 atoms, more often from about 13 atoms to about 25 atoms, and most often from about 13 atoms to about 20 atoms.

Exemplary linkers that can be used in methods and compositions of the disclosure include, but are not limited to, polyethylene glycol (PEG), polyvinyl acid (PVA), polycaprolactone (PCL), polylactic acid (PLA), poly(lactic-co-glycolic acid) (PLGA), polyurethane (PU), polyglycolic acid (PGA), polyethylene oxide (PEO), polypeptide chain, aliphatic chain, or a combination thereof.

As used herein, the terms “polypeptide” and “polypeptide chain” when referring to a linker are used interchangeably herein and refer to two or more amino acids that are linked together. Polypeptide can also include substitution on the side chain of the amino acids, e.g., methylated or demethylated lysine, methylated cysteine, etc. In general, a suitable polypeptide linker is one that does not elicit any significant immune response from a subject to which the surface modified immune cell is administered.

The term “aliphatic chain” means hydrocarbon chain having at least two carbon chain atoms, typically at least 4 carbon chain atoms, often at least 6 carbon chain atoms, more often at least 8 carbon chain atoms, and most often at least 10 carbon chain atoms. The aliphatic chain can optionally include a carbon-carbon double bond, carbon-carbon triple bond, a cyclic carbon having from about three to twenty carbon atoms, typically from about three to about twelve carbon atoms, often from about three to ten carbon atoms, and most often three to about eight carbon atoms in the cyclic ring structure. The cyclic ring structure can be saturated (e.g., cyclopentyl, cyclohexyl, dodecyl, norbornyl, etc.) or can have one or more unsaturated carbon-carbon bonds, or can be aromatic (e.g., phenyl, naphthyl, anthracyl, etc.) or can have one or more heteroatoms (e.g., O, NR′, where R′ is H or C₁-C₂₀ alkyl, S(O)n, where n is 1 or 2) within the cyclic structure. The aliphatic chain can optionally be substituted with one or more substituents, where each substituent can independently be ether (e.g., —OR′, where R′ is alkyl), halide, protected amine (e.g., dimethyl amino, etc.), cyano, nitro, nitroso, sulfate, phosphate, etc.

Still in other embodiments, the immune cell comprises a T cell, B cell, natural killer (NK) cell, macrophage, neutrophil, dendritic cell, mast cell, eosinophil, or basophil. In one particular embodiment, the immune cell comprises a T cell. Still in other embodiments, the immune cell is γδT cell (i.e., gdT cell).

Unlike conventional immune therapy where autologous immune cells are often used, methods and compositions of the disclosure can include allogeneic immune cells. Accordingly, in some embodiments, immune cells are collected from a plurality of healthy donors, e.g., a pooled immune cells from a plurality of donors is used. As used herein, the term “healthy donors” and grammatical variations thereof refers to a donor who does not have the clinical condition or disease to be treated in a subject.

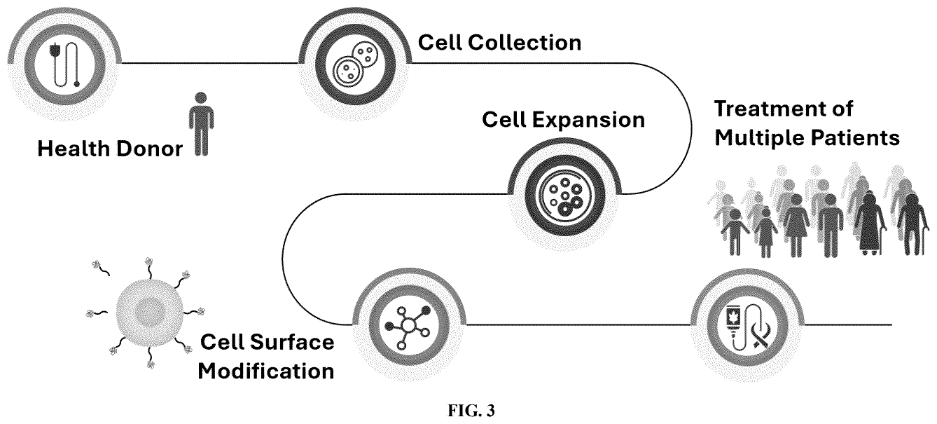

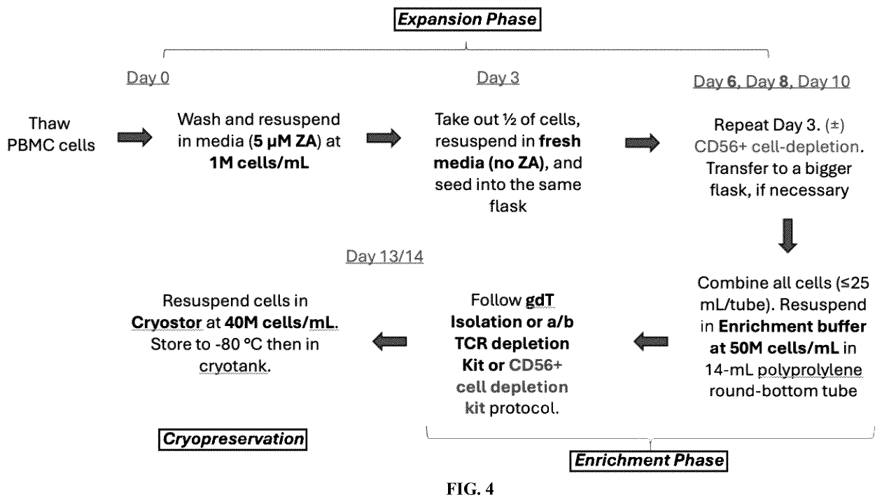

For brevity and clarity, one exemplary method for producing surface modified immune cells of the disclosure is illustrated as follows. Peripheral blood mononuclear (PBMC) cells are obtained from the subject (autologous cells), or a healthy donor (FIG. 3) or a plurality of healthy donors (allogeneic cells). The cells are expanded under appropriate conditions known to one skilled in the art. One particular condition for expanding PBMC cells uses appropriate media known to one of ordinary skill in the art supplemented with human IL-2 and/or IL-15. See, FIG. 4 (exemplary media used is CTS Optimizer T cell expansion media (w/supplement), 10 pg/mL Gentamicin, 2% FBS, 1000 IU/mL Human IL-2, 100 IU/mL IL-15 5 u M Zoledronic Acid (ZA). Cells can be collected at certain stages and analyzed using flow cytometer). After a sufficient number of PBMC cells have been obtained, cells are purified and harvested. In one particular embodiment, gdT cells are isolated for surface modification. In this manner, surface modified immune cells of the disclosure can be readily expanded and prepared, thereby allowing treatment of multiple subjects or patients.

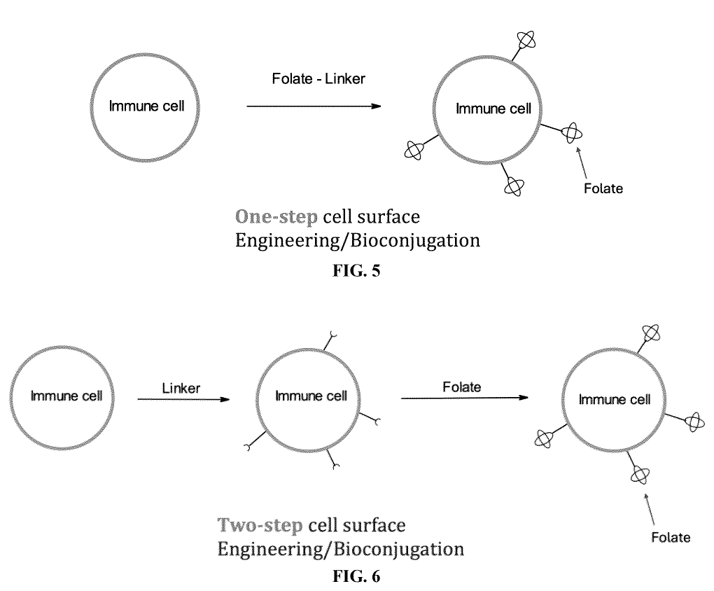

Isolated gdT cells can be surface modified using any of the methods disclosed herein. Two such methods are schematically illustrated in FIGS. 5 and 6. In FIG. 5, a solution of ligand-linker-NHS ester (e.g., “Folate-Linker” in FIG. 5) is added to the isolated gdT cells and incubated under conditions sufficient to attach or covalently bind NHS ester portion of the ligand-linker-NHS ester compound to a functional group that is present on the surface of the immune cell. The amount of reaction time can vary depending on a variety of factors such as concentration of the ligand-linker-NHS ester compound, the nature of the immune cell (e.g., gdT cell, NK cell, neutrophil, dendritic cell, mast cell, eosinophil, basophil, etc.), temperature of the mixture, the number or amount of ligand-linker-NHS ester attachment or binding desired, etc. Generally, the surface modification is conducted at a temperature of from about 10° C. to about 30° C., typically from about 15° C. to about 25° C., and often at room temperature. The amount of reaction time required for covalently attaching the ligand-linker-NHS ester compound also depends on a variety of factors such as those discussed above. Typically, the reaction time ranges from about 10 min to about 120 min, often from about 20 min to about 60 min, and more often from about 30 min to about 45 min. It should be appreciated, however, the scope of the disclosure is not limited to these temperature ranges and/or time ranges. As stated above, reaction conditions can vary from those disclosed herein. Discussion of particular reaction conditions is provided solely for the purpose of illustrating the practice of the disclosure and do not constitute limitations on the scope thereof. The surface modified cells are then isolated and purified. The density or the amount of surface modified immune cells are then adjusted to the desired level for use.

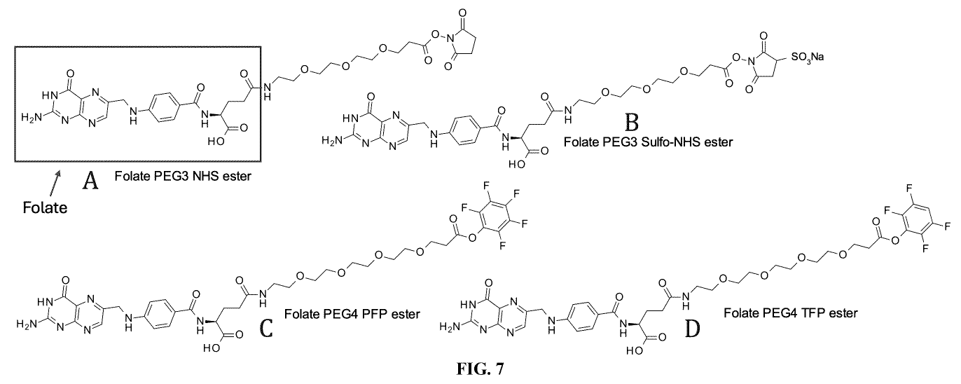

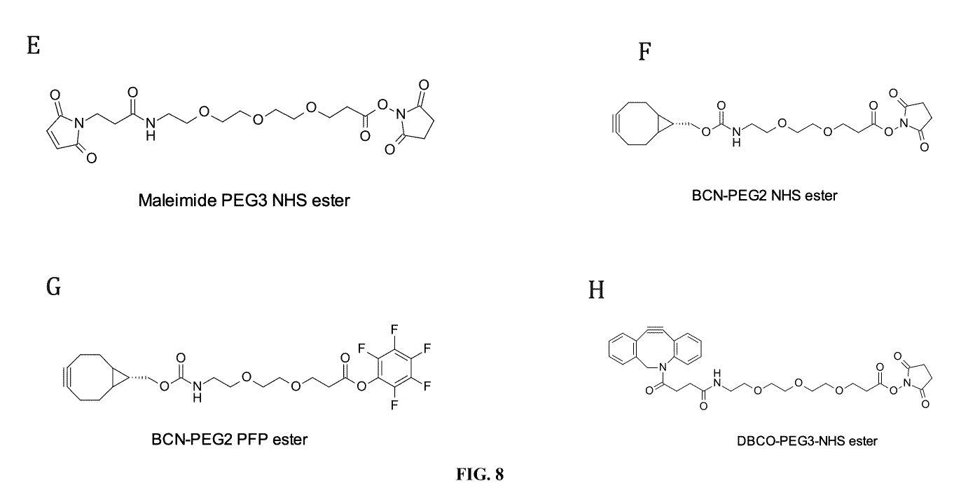

More generally, surface modified immune cells can be produced by reacting the desired immune cells with a compound of the general formula: A-Bₓ-C (where A is a ligand that is capable of binding selectively to the target cell, B is a linker, x is 0 or 1 (i.e., linker may be optional), and C is a functional group capable of reacting with a complementary functional group that is present in the immune cell surface. The functional group “C” can be N-hydroxysuccinimide ester as illustrated above. Other useful “C” functional groups include, but are not limited to, anhydride, acyl chloride, pentafluorophenyl ester, tetrafluorophenyl ester, isocyanate, as well as other suitable functional groups known to one skilled in the art. Exemplary folate and linkers and functional groups used in surface modification of immune cells are shown in FIG. 7. As used herein, the term “treating”, “contacting” or “reacting” when referring to producing a surface modified immune cell means adding or mixing two or more reagents along with the immune cell under appropriate conditions to produce the surface modified immune cell. It should be appreciated that the reaction which produces the indicated and/or the desired product may not necessarily result directly from the combination of two reagents which were initially added, i.e., there may be one or more intermediates which are produced in the mixture which ultimately leads to the formation of the surface modified immune cells.

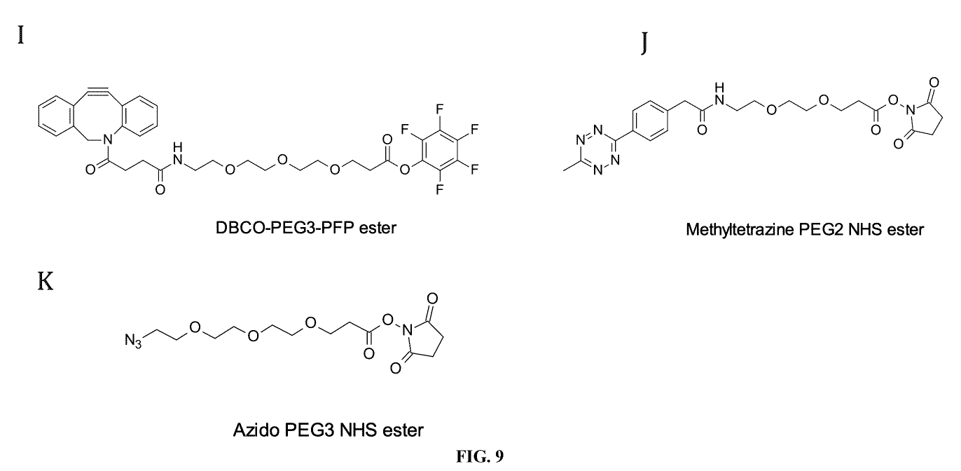

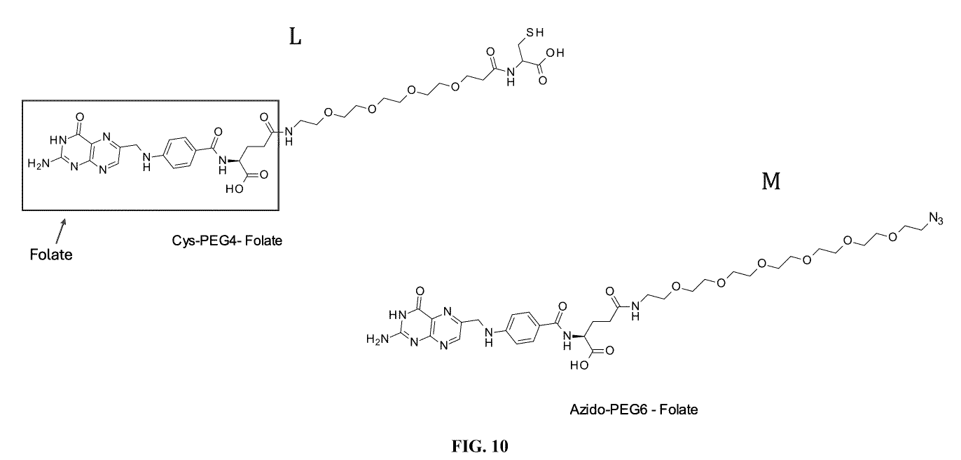

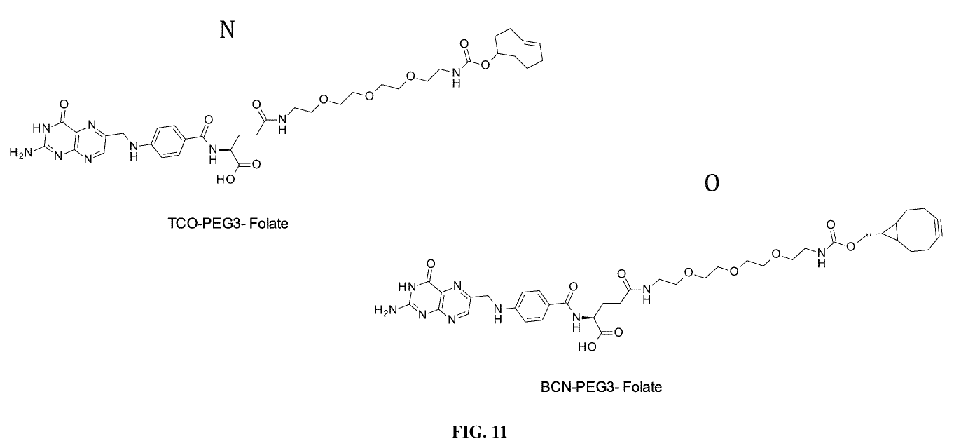

Alternatively, as illustrated in FIG. 6, the surface modified immune cells of the disclosure can be produced by reacting a linker, i.e., a compound of the general formula: A′-Bₓ-C (where A′ is a functional group that is capable of reacting with a complementary functional group that is present in a ligand, B is a linker, x is 0 or 1 (i.e., linker is optional), and C is a functional group capable of reacting with a complementary functional group that is present in the immune cell surface. In this manner an intermediate surface modified immune cell of the general formula: A′-Bx-C′-Q is produced, where A′, B, and x are those defined herein, and Q is an immune cell in which a moiety of the formula A′-Bx is attached, and C′ is a resulting functional group from the reaction of C with the complementary functional group on the immune cell surface. For example, if C is an NHS ester functional group that reacts with an amine group that is present on the immune cell surface, the resulting C′ would be an amide functional group (e.g., a moiety of the formula —C(═O)—NH—). This intermediate surface modified immune cell can then be reacted with a ligand (Lg) having a suitable complementary functional group that can react with the functional group that is present in A′ to produce a surface modified immune cell. Exemplary linkers and functional groups for this two-step process for producing surface modified immune cells are illustrated in FIGS. 8-11.

It should be appreciated that the scope of the disclosure is not limited to these two methods for producing surface modified immune cells. Other variations of reaction can also be used to produce the surface modified immune cells of the disclosure. For example, one can react A-B′ with C″-Q, where A and Q are as defined herein and B′ is a linker with a functional group that reacts with a complementary functional group C″ that is present on the immune cell (Q) surface. In this manner, one can produce surface modified cells using a variety of strategies.

Unlike many conventional surface modified immune cell therapies, compositions and methods of the disclosure provide surface modified immune cells that are covalently linked to a ligand (e.g., folate or a derivative thereof) via a linker to increase selectivity and/or specificity to a desired target cell.

In this manner the surface modified immune cells of the disclosure can be used in a variety of therapeutic applications including, but not limited to, treating tumor or cancer, treating an inflammatory disease, treating an infection, treating an autoimmune disease, and treating an immunodeficiency disease, cardiometabolic diseases, and clinical conditions or diseases associated with senescence, such as senility, Alzheimer's disease, and other cognitive impairments, etc. In some embodiments, the surface modified immune cells are used to treat a tumor or cancer. Exemplary cancers that can be treated using surface modified immune cells of the disclosure include, but are not limited to, ovarian cancer, breast cancer, lung cancer, stomach cancer, colorectal cancer, brain cancer, melanoma, sarcoma, lymphoma, blastoma, carcinosarcoma, carcinoma, kidney cancer, bladder cancer, pancreatic cancer, cervical cancer, uterine cancer, and other solid tumor cancers known to one skilled in the art. In one particular embodiment, the cancer is a blood cancer. Still in other embodiments, the cancer is multiple myeloma, hematopoietic malignancies of myeloid origin, such as myelogenous leukemias. Yet in other embodiments, the cancer is a solid cancer.

Exemplary inflammatory diseases that can be treated using surface modified immune cells of the disclosure include, but are not limited to, Autoimmune disease, gastrointestinal disease, neurodegenerative disease, as well as other inflammatory diseases known to one skilled in the art.

Exemplary infections that can be treated using surface modified immune cells of the disclosure include, but are not limited to, viral infection such as SARS-COV, HBV, and other infections due to bacterium, virus, protozoa, mycobacterium, fungus, or other microorganism infections.

Exemplary autoimmune diseases that can be treated using surface modified immune cells of the disclosure include, but are not limited to, Rheumatoid arthritis, osteoarthritis, systemic Lupus Erythematosus, sclerosis, psoriasis, as well as other autoimmune diseases known to one skilled in the art, or a combination thereof.

Exemplary immunodeficiency diseases that can be treated using surface modified immune cells of the disclosure include, but are not limited to, HIV, as well as other immunodeficiency diseases known to one skilled in the art.

Surface modified immune cells of the disclosure can be used alone or in combination with other therapeutically active agents and/or treatment methods. For example, one particular aspect of the disclosure includes a method for treating cancer in a patient using surface modified immune cells of the present disclosure in combination with a conventional cancer treatment. In the context of the present disclosure, it is contemplated that this combined treatment can include, but is not limited to, chemotherapeutic, radiation, a polypeptide inducer of apoptosis, or other therapeutic intervention. The scope of the present disclosure also includes using more than one administration of the treatment. In some embodiments for treating cancer, surface modified immune cells of the disclosure are used in combination with other anticancer treatments such as chemotherapeutic agents, ionizing radiation, hormonal therapy, cytokines, immunotherapy, cellular therapy, vaccines, monoclonal antibodies, antiangiogenic agents, targeted therapeutics (small molecule drugs), or biological therapies. For example, chemotherapeutic agents that can be used in addition to surface modified immune cells of the disclosure include, but are not limited to, antitumor alkylating agents such as Mustards (mechlorethamine HCl, melphalan, chlorambucil, cyclophosphamide, ifosfamide, busulfan), Nitrosoureas (BCNU/cannustine, CCNU/lomustine, MeCCNU/semustine, fotemustine, treptozotocin), Tetrazines (dacarbazine, mitozolomide, temozolomide), Aziridines (thiotepa, mitomycin C, AZQ/diaziquone), procarbazine HCl, hexamethylmelamine, adozelesin; cisplatin and its analogues, cisplatin, carboplatin, oxaliplatin; antimetabolites, methotrexate, other antifolates, 5-fluoropyrimidines (5-fluorouracil/5-FU), cytarabine, azacitidine, gemcitabine, 6-thiopurines (6-mercaptopurine, thioguanine), hydroxyurea; topoisomerase interactive agents epipodophyllotoxins (etoposide, teniposide), camptothecin analogues (topotecan HCl, irinotecan, 9-aminocamptothecin), anthracyclines and related compounds (doxorubicin HCl, liposomal epirubicin, daunorubicin HCl, daunorubicin HCl citrate liposomal, epirubicin, idarubicin), mitoxantrone, losoxantrone, actinomycin-D, amsacrine, pyrazoloacridine; antimicotubule agents Vinca alkaloids (vindesine, vincristine, vinblastine, vinorelbine), the taxanes (paclitaxel, docetaxel), estramustine; fludarabine, 2-chlorodeoxyadenosine, 2′-deoxycoformycin, homoharringtonine, suramin, bleomycin, L-asparaginase, floxuridine, capecitabine, cladribine, leucovorin, pentostatin, retinoids (all-trans retinoic acid, 13-cis-retinoic acid, 9-cis-retinoic acid, isotretinoin, tretinoin), pamidronate, thalidomide, cyclosporine; hormonal therapies antiestrogens (tamoxifen, toremifene, medroxyprogesterone acetate, megestrol acetate), aromatase inhibitors (aminoglutethimide, letrozole/femara, anastrozole/arimidex, exemestane/aromasin, vorozole), gonadotropin-releasing hormone analogues, antiandrogens (flutamide, casodex), fluoxymeterone, diethylstilbestrol, octreotide, leuprolide acetate, zoladex; steroidal and non-steroidal anti-inflammatory agents (dexamethasone, prednisone); Monoclonal antibodies including, but not limited to, anti-HER2/neu antibody (herceptin/trastuzumab), anti-EGFR antibody (cetuximab/erbitux, ABX-EGF/panitumumab, nimotuzurnab), anti-CD20 antibody (rituxan/rituximab, ibritumomab/Zevalin, tositumomab/Bexxar), anti-CD33 antibody (gemtuzumab/MyloTarg), alemtuzumab/Campath, bevacizumab/A vastin; and small molecule inhibitors.

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiation therapy used according to the present disclosure can include, but is not limited to, the use of γ-rays, X-rays, positrons, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy can include the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area. Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed.

Still in other embodiments, methods of the disclosure can include using a radiosensitizer to make the tumor cells more likely to be damaged, or radioprotectors to protect normal tissues from the effects of radiation. Yet in other embodiments, methods of the disclosure can include using hyperthermia in sensitizing tissue to radiation.

In yet other embodiments, administration of surface modified immune cells of the disclosure can be combined with other secondary therapies for treating cancer. Exemplary secondary cancer treatments or therapies include those discussed above, as well as surgery, gene therapy, miRNAs, as well as other agents such as immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents.

Another type of therapy used in treating glioblastoma patients is Tumor Treating Fields (TTF). TTF is administered in the home or outpatient setting and involves wearing scalp transducers on the shaved head to deliver alternating electrical fields, with frequent reshaving and reapplying transducers. The administration of the compounds of the disclosure can be combined with TTF.

As used herein, the terms “treatment,” “treating” and other grammatical variations thereof refers to an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” along with grammatical variations thereof are used interchangeably herein to describe a mammal, including humans. In some embodiments, the individual is human. In some embodiments, an individual suffers from a disease or condition (e.g., cancer). In some embodiments, the individual is in need of treatment.

As is understood in the art, an “effective amount” refers to an amount of an agent (e.g., a targeting conjugate) sufficient to produce a desired therapeutic outcome (e.g., reducing the severity or duration of, stabilizing the severity of, or eliminating one or more symptoms of cancer) or a desired diagnostic outcome. For therapeutic use, beneficial or desired results include, e.g., decreasing one or more symptoms resulting from the disease (biochemical, histologic and/or behavioral), including its complications and intermediate pathological phenotypes presented development of the disease, increasing the quality of life of those suffering from the disease, decreasing the dose of other medications required to treat the disease, enhancing effect of another medication, delaying the progression of the disease, and/or prolonging survival of patients. In some embodiments, an effective amount of the agent may extend survival (including overall survival and progression free survival) result in an objective response (including a complete response or a partial response); relieve to some extent one or more signs or symptoms of the disease or condition; and/or improve the quality of life of the subject.

Further provided by the present disclosure are compositions (e.g., pharmaceutical compositions), kits, and articles of manufacture comprising surface modified immune cells disclosed herein. In some embodiments, there is provided a pharmaceutical composition comprising surface modified immune cells described herein and a pharmaceutically-acceptable carrier. In some embodiments, the composition (such as pharmaceutical composition) comprises a carrier, diluent, or excipient, which may facilitate administration of the composition to an individual in need thereof. Examples of carriers, diluents, and excipients include, but are not limited to, sodium chloride, calcium carbonate, calcium phosphate, amino acids, various sugars such as lactose, or types of starch, cellulose derivatives, gelatin, vegetable oils, polyethylene glycols and physiologically compatible solvents.

In some embodiments, the pharmaceutical composition comprises autologous surface modified immune cells. Still in other embodiments, the pharmaceutical composition comprises allogeneic surface modified immune cells. Yet in further embodiments, the pharmaceutical composition comprises a plurality of allogeneic surface modified immune cells, i.e., surface modified immune cells produced from a plurality of donors. In some embodiments, surface modified immune cells are obtained from at least about five, typically from at least about ten, often from at least about fifty, more often from at least about one hundred, still more often from at least about two-hundred fifty, even more often from at least five-hundred, and most often from at least one-thousand donors.

The pharmaceutical compositions described herein can be prepared by mixing surface modified immune cells of the disclosure along with other pharmaceutically acceptable excipients, carriers, or ingredients following generally accepted procedures. For example, the selected components may be simply mixed in a standard device to produce a concentrated mixture which may then be adjusted to the final concentration and viscosity by the addition of water or thickening agent and possibly a buffer to control pH or an additional solute to control tonicity.

Other pharmaceutically acceptable carriers and their formulation are described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42: 2S (1988).

In some embodiments, the pharmaceutical composition is a liquid suspension. In some embodiments, the pharmaceutical composition is a sterile composition.

Also provided are kits comprising surface modified immune cells described herein. The kits of the disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present disclosure thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating or diagnosing a disease as described herein, and may have a sterile access port. The label or package insert indicates that the composition is used for treating or diagnosing a disease in an individual. The label or package insert will further comprise instructions for administering the composition to the individual.

Package insert refers to instructions customarily included in commercial packages of therapeutic products that contain information about the indications, usage, dosage, administration, contraindications and/or warnings concerning the use of such therapeutic or diagnostic products. In some embodiments, the package insert indicates that the composition is used for treating or diagnosing a disease (such as cancer).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Additional objects, advantages, and novel features of this disclosure will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Example 1. This example illustrates one particular method for gdT cell expansion.

gdT cells were expanded from cryopreserved PBMC cells with the use of Zoledronic acid and supplementation with human IL-2 and IL-15. Briefly, as shown in FIG. 4 flow chart, a vial of cryopreserved PBMC cells (50-100 M) was thawed and seeded in T-225 flasks at 1×10⁶ cells/mL in CTS OpTMizer T cell expansion media (Cat #A1048501, Gibco) supplemented with Gentamicin, 2% FBS, 2 mM Gln, 1000 IU/mL human IL-2, 100 IU/mL human IL-15, 70 μM Ascorbic acid and 5 μM Zoledronic acid (ZA). On Day 3, 50% of the media was taken out and replenished with fresh one containing all the ingredients stated above but without ZA. This process was repeated on Day 6. On Day 8, 50% of media was taken out and replenished with fresh media in the amount corresponding to a seeding density of 1.2-1.5×10⁶ cells/mL, increasing the number of flasks as needed. This process was repeated on Days 10 and 12. Cells were harvested on Day 13-14 where the gdT cells have expanded to about a thousand-fold and the purity was approximately 90%. Cells are further subjected to either one of the following if higher % purity is desired: (1) EasySep™ Human gdT Cell Isolation kit (Cat #19255, Stemcell Technologies), (2) EasySep™ Human TCR a/b Depletion kit (Cat #17847, Stemcell Technologies), or (3) CD56 Microbeads/LS Column/MidiMac Separator Kit (130-050-401, Miltenyi Biotec).

Example 2. This example illustrates one particular embodiment of gdT cell expansion in the absence of antigen.

gdT cells (specifically Vγ9Vδ2) were expanded from cryopreserved human peripheral blood mononuclear cells (PBMC) for 12-14 days with the use of Zoledronic acid (ZA) in serum-free media supplemented with human IL-2. Briefly, a vial of PBMC containing 80-120 M cells was thawed and seeded in non-tissue treated T-225 flasks at 1.5-2×10⁶ cells/mL in TheraPEAK T-VIVO medium (Cat #BP12-970Q, Lonza) supplemented with 100 IU/mL human IL-2 and 6 μM ZA and left in 37° C. incubator with 5% CO₂. On Days 2 and 4, 50% of the media was taken out and replenished with fresh media containing 100 IU/mL human IL-2 (expansion media). On Day 6, the cell culture was either depleted with alpha beta T cells or not. If depleted, the EasySep™ Human TCR a/b Depletion kit (Cat #17847, Stemcell Technologies) was utilized following the manufacturer's protocol with reduced amount of the antibody cocktail. After depletion, cells were transferred into a shake flask with a seeding density of 0.8-1×10⁶ cells/mL resuspended in the expansion media and back to the incubator with gentle agitation. Cells that are not depleted are simply transferred to a shake flask after dilution with fresh media and back to the incubator with gentle agitation. On Days 7-8, the cell culture was diluted as needed with the expansion media to 2×10⁶ cells/mL and starting on Day 9, 50% of the cell culture was spun down and seeded back into the original flask by replenishing with fresh expansion media adjusting the overall cell density to 2×10⁶ cells/mL. Cells were diluted the next day to the same density. The processes done on Days 9-10 were repeated until the day the cells are harvested. Cells were harvested starting on Day 10 with approximate purity of >85-98%. Cells are further subjected to either one of the following if higher % purity is desired: (1) EasySep™ Human gdT Cell Isolation kit (Cat #19255, Stemcell Technologies), (2) EasySep™ Human TCR a/b Depletion kit (Cat #17847, Stemcell Technologies), or (3) CD56 Microbeads/LS Column/MidiMac Separator Kit (130-050-401, Miltenyi Biotec). % purity of resulting gamma delta T cells was assessed using flow cytometer.

Example 3. This example illustrates one method for gdT cell surface conjugation.

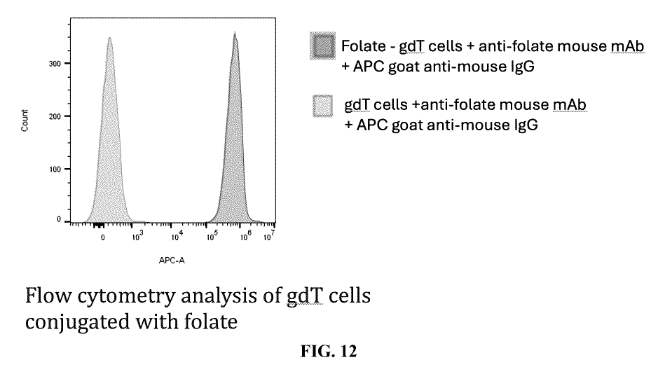

gdT cells designated for conjugation were counted and tested for viability. As illustrated in FIG. 5, to the cells were added 20 μM folate PEG3 NHS ester and incubated at room temperature for 30 minutes in the dark. After incubation, unreacted folate compound was removed through washing steps. The cell density of the conjugated gdT cells was adjusted to the desired level for use. Additionally, two control groups were prepared: one with unconjugated gdT cells as a control, and the other with gdT cells spiked with 20 μM folic acid. Flow cytometry analysis of gdT cells conjugated with folate is shown in FIG. 12. As can be seen, this method provides a highly efficient attachment of folate-linker conjugate to gdT cell surface.

Example 4. This example illustrates conjugation of folate to gdT cells in a two-step process as illustrated in FIG. 6.

In the first step, the alkyne-containing linker was attached to the surface of the cells. This was followed by the strain-promoted azide-alkyne reaction (i.e., click chemistry) between the gdT-linker and the azide-containing folate reagent. Prior to the chemical reaction, gdT cells designated for conjugation were counted and tested for viability. To the cells were added 20-200 μM DBCO NHS ester and incubated for 30 min at room temperature in the dark with gentle agitation. Cells were washed thoroughly after the reaction and continued to react with the azido-Folate reagent with concentrations ranging from 50-1000 M for 2 h at 37° C. with gentle agitation. Finally, cells were washed thoroughly in tumor cell growth media and resuspended into the desired concentration for further analysis. Conjugation of folate to gdT cells was confirmed via flow analysis.

Example 5. This example illustrates evaluation of binding of folate conjugated on γδT cell surface and human folate receptors.

To estimate the binding between Folate conjugated on γδT cell surface and human folate receptors, 1 μg/mL human folate receptor alpha (hFOLR1, human Fc tag)/human folate receptor beta (hFOLR2, His Tag) recombinant proteins were co-incubated with 100,000 γδT cells either non-conjugated or conjugated with Folate in 100 μl FACs buffer (DPBS with 2% heat-inactivated FBS) for 30 min at room temperature (RT). Cells were spun down at 350×g for 5 min at RT to remove the supernatant. After cells were washed one more time with 100 μl FACs buffer, the cells were resuspended in FACs buffer containing either PE-Anti-Fc or APC-Anti-His mAbs (1:400 dilution) together with 7-AAD (2.5 μg/ml). After 15 min incubation in dark at RT, cells were spun down as described above and washed once with 100 μl FACs buffer. Cells were resuspended in 100 μl/well FACS buffer and analyzed by using Cytek spectrum flowcytometry.

### Results

As shown in FIG. 22, significant amounts of hFOLR1 and hFOLR2 protein are detected on the Folate conjugated γδT cells, but not on non-conjugated γδT cells. This clearly shows folate is target ligand for hFOLR1 and hFOLR2 that are overexpressed in tumor cells.

Example 6. This example shows Granzyme B upregulation in Folate-Conjugated γδT cells following tumor cell-mediated activation. (Mechanism study. Granzyme-protein biomarker by T cells. Upregulates tumor cells. E=immune or Effector cell; T=target or tumor cell).

To assess the activation and cytotoxic potential of folate-conjugated γδT cells, upregulation of Granzyme B upon co-culture with tumor cells was quantified using ELISA. THP-1 tumor cells were resuspended at a concentration of 0.5×10⁶ cells/mL in complete tumor culture medium, and 100 μl of this suspension was added to each well of a non-tissue culture-treated 96-well plate. Folate-conjugated and non-conjugated γδT cells were adjusted to a concentration of 3×10⁶ cells/ml in complete tumor culture medium, and one 3-fold serial dilution followed by two 5-fold consecutive dilutions were performed in a 96-well plate. Subsequently, 50 μl of γδT cells from each dilution were added to the wells containing THP-1 cells, resulting in E:T (immune cell and target or tumor cell, respectively) ratios of 1:1, 1:5, and 1:25. The folate-conjugated and non-conjugated γδT cells only controls were set up the same as above, but without any THP-1 cells in the wells. The experimental and control groups were conducted in triplicate. The plates were then transferred to a humidified tissue culture incubator at 37° C. with 5% CO₂.

After overnight incubation, the plates were centrifuged at 350×g for 5 minutes at room temperature, and the supernatant was harvested. 50 ul supernatant of each sample was taken to evaluate Granzyme B expression level in supernatant by using Human Granzyme B DuoSet ELISA kit (R&D System, CAT #DY2906-05) according to manufacture manual. Briefly, samples and standards were added to the plate and incubated for 2 h. After washing, detection antibody was introduced for another 2h incubation. The plate was washed again, followed by the addition of a Streptavidin-HRP enzyme conjugate and a 20 min incubation in the dark. Following a final wash, substrate solution was added, which was terminated with a stop solution, and the optical density was immediately measured with a microplate reader at 450 nm, with wavelength correction at 540 nm. The Granzyme B protein expression level was calculated using the generated standard curve.

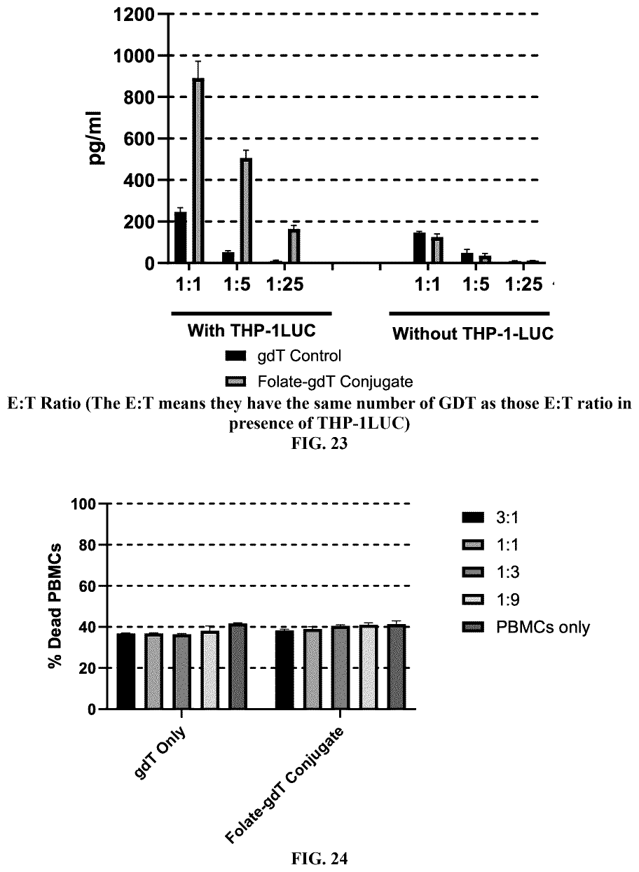

To assess target-specific activation, folate-conjugated γδT cells (effector cells) were co-cultured with folate receptor (FOLR)-positive THP-1LUC tumor cells (target cells) at various effector-to-target (E:T) ratios. As shown in FIG. 23, this co-incubation resulted in a substantial, E:T ratio-dependent secretion of Granzyme B, a key mediator of cytotoxicity, into the culture supernatant. Specifically, Granzyme B concentrations reached 891.14 pg/ml (1:1 E:T), 506.01 pg/ml (1:5 E:T), and 164.25 pg/ml (1:25 E:T). In contrast, non-conjugated γδT cells released significantly lower amounts of Granzyme B under the same conditions (246.67 pg/ml, 52.18 pg/ml, and 10.39 pg/ml at 1:1, 1:5, and 1:25 E:T ratios, respectively). In the absence of THP-1LUC target cells, both conjugated and non-conjugated γδT cells exhibited minimal, comparable basal levels of Granzyme B secretion.

### Results

These data demonstrate that folate conjugation significantly enhances the target-directed activation of γδT cells against FOLR-expressing cancer cells, leading to a marked increase in cytotoxic potential.

Example 7. This example is directed to evaluating cytotoxicity of folate conjugated γδT cells.

The cytotoxicity assay was conducted using flow cytometry. Effector cells included folate-conjugated γδT cells (one step conventional), non-conjugated γδT cells, and folate-conjugated γδT cells (via click chemistry). The target is THP-1 tumor cells.

To differentiate between effector and target cells, the tumor cells were pre-stained with 1 μM CFSE in DPBS for 10 minutes at room temperature in the dark. Excess CFSE was then removed by washing the cells twice with complete tumor culture medium (folic acid-free RPMI 1640 containing 10% HI-FBS). CFSE-labeled tumor cells were resuspended at a concentration of 0.5×10⁶ cells/mL in complete tumor culture medium, and 100 μl of this suspension was added to each well of a non-tissue culture-treated 96-well plate. Folate-conjugated γδT cells, non-conjugated γδT cells and folate-conjugated γδT cells (click chemistry) were adjusted to a concentration of 1×10⁶ cells/ml in complete tumor culture medium, and three consecutive 5-fold serial dilutions were performed in a 96-well plate. Subsequently, 50 μl of γδT cells from each dilution were added to the wells containing tumor cells, resulting in E:T ratios of 1:1, 1:5, 1:25, and 1:125. All experimental and control groups were set up in triplicate. The plates were then transferred to a humidified tissue culture incubator at 37° C. with 5% CO₂.

After overnight incubation, the plates were centrifuged at 300×g for 5 minutes at room temperature, and the supernatant was discarded. Cells in each well were stained with 30 μl of DPBS containing live/dead fixable yellow dye (diluted 1:500) for 10 minutes at room temperature in the dark. Following staining, 150 μl of FACs buffer (DPBS with 2% HI-FBS) was added to each well, the plates were centrifuged again at 300×g for 5 minutes at room temperature. After removing the supernatant, the cells were stained with 30 μl of Annexin V binding buffer mixed with 1:50 diluted anti-Annexin V monoclonal antibody labeled with Alexa Fluor 647 for 20 minutes at room temperature in dark. After staining, 30 μl of the sample was collected for flow cytometry analysis, with 50 μl of Annexin V binding buffer added to each well (30 μl out of a total 80 μl volume). Viable tumor cells were defined as double-negative for both live/dead yellow and Annexin V staining. The percentage of apoptotic or dead tumor cells was determined based on Annexin V-positive and/or live/dead positive staining.

### Results

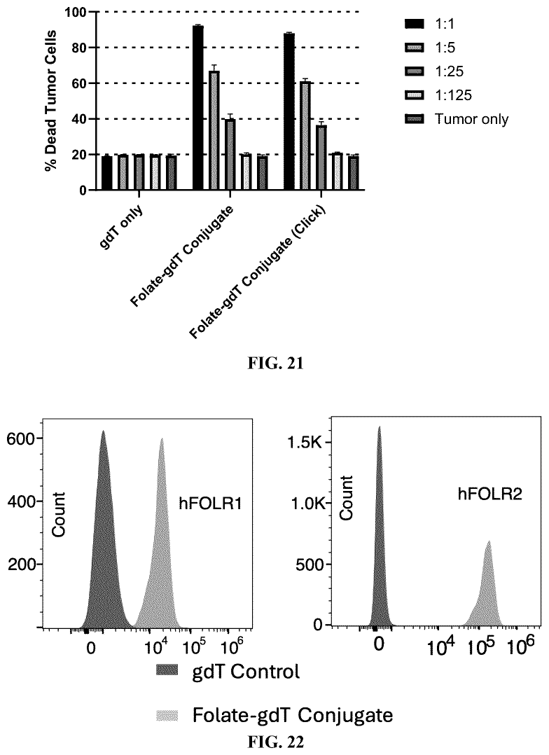

As shown in FIG. 21, folate-conjugated γδT cells (click) exhibited potent and dose-dependent cytotoxicity against THP-1 target cells. At a 1:1 E:T ratio, γδT cells conjugated via two different methods (henceforth NHS and Click) induced approximately 92% and 88% tumor cell lysis, respectively. As the E:T ratio was reduced to 1:5, 1:25, and 1:125, respectively, the efficacy of folate-conjugated γδT cells decreased to about 67%, 39%, and 20%, and that of folate-conjugated γδT cells (Click) dropped approximately to 61%, 36% and 21%. In contrast, non-conjugated γδT cells did not exhibit significant cytotoxicity against THP-1 cells, demonstrating that the observed cytotoxicity was dose-dependent and specifically induced by both folate-conjugated γδT cells (NHS or Click).

Example 8. This example is directed to evaluating cytotoxity of folate conjugated gdT cells against peripheral blood mononuclear cells (PBMCs). (PBMC as control; gdT=γδT cells)

The cytotoxicity assay was conducted using flow cytometry. Effector cells included folate-conjugated γδT cells and non-conjugated γδT cells. The target cells are PBMCs purified from leukopak collected from a healthy donor.

To differentiate between effector and target cells, the PBMCs were pre-stained with 1 μM CFSE in DPBS for 10 minutes at room temperature in the dark. Excess CFSE was then removed by washing the cells twice with complete tumor culture medium (folic acid-free RPMI 1640 containing 10% HI-FBS). CFSE-labeled PBMCs were resuspended at a concentration of 0.5×10⁶ cells/mL in complete tumor culture medium, and 100 μl of this suspension was added to each well of a non-tissue culture-treated 96-well plate. Folate-conjugated and non-conjugated γδT cells were adjusted to a concentration of 3×10⁶ cells/mL in complete tumor culture medium, and three consecutive 3-fold serial dilutions were performed in a 96-well plate. Subsequently, 50 μl of γδT cells from each dilution were added to the wells containing PBMCs, resulting in E:T ratios of 3:1, 1:1, 1:3, and 1:9. All experimental and control groups were set up in triplicate. The plates were then transferred to a humidified tissue culture incubator at 37° C. with 5% CO₂.

After overnight incubation, the plates were centrifuged at 350×g for 5 minutes at room temperature, and the supernatant was discarded. Cells in each well were stained with 30 μl of DPBS containing live/dead fixable yellow dye (diluted 1:500) for 10 minutes at room temperature in the dark. Following staining, 150 μl of FACs buffer (DPBS with 2% HI-FBS) was added to each well, and the plates were centrifuged again at 350×g for 5 minutes at room temperature. After removing the supernatant, the cells were stained with 30 μl of Annexin V binding buffer mixed with 1:50 diluted anti-Annexin V monoclonal antibody labeled with Alexa Fluor 647 for 20 minutes at room temperature in the dark. After staining, 30 μl of the sample was collected for flow cytometry analysis, with 50 μl of Annexin V binding buffer added to each well (30 μl out of a total 80 μl volume). Viable PBMCs were defined as double-negative for both live/dead yellow and Annexin V staining. The percentage of apoptotic or dead PBMCs was determined based on Annexin V-positive and/or live/dead positive staining.

### Results

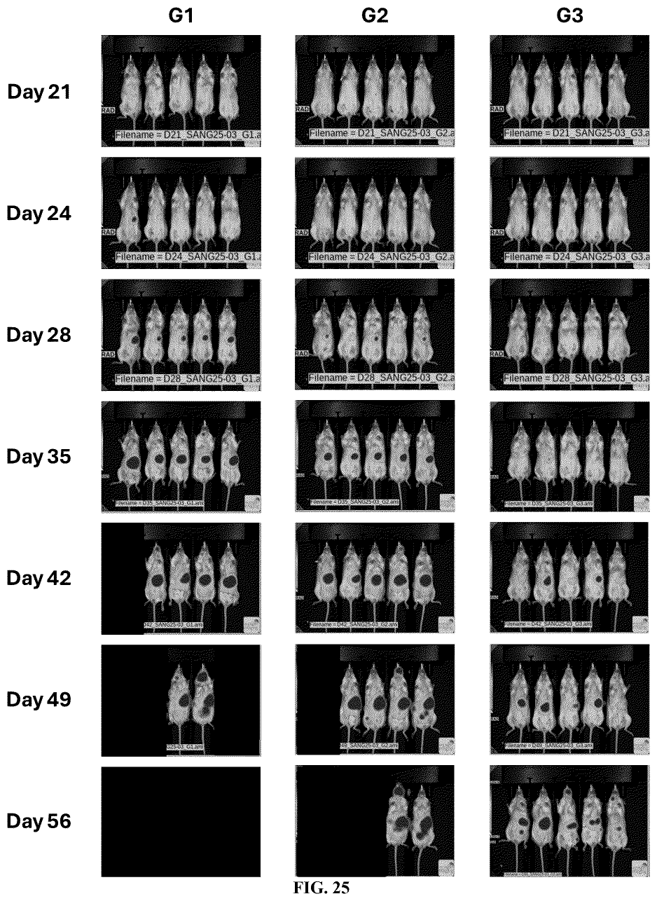

Following overnight incubation, PBMCs cultured in medium alone demonstrate a basal non-viability of approximately 40%. As shown in FIG. 24, co-incubation with either folate-conjugated or non-conjugated γδT cells, across E:T ratios from 3:1 to 1:9, do not result in a statistically significant increase in PBMC mortality when compared to the PBMC monoculture control. These results indicate that neither conjugated nor non-conjugated γδT cells induce cytotoxicity against normal PBMCs, suggesting they are safe for non-target cells. (E/T ratio. E=Effector (immune) cell, T=target (PBMC) cells. Shows “normal cell, i.e., control” is not effected. Therefore, safe)

Example 9. This example is directed to determining cytotoxicity of folic acid conjugated gdT cells on various tumor cells.

### Materials:

Annexin V staining buffer: Biolegend, Cat #422201
Annexin V antibody: Biolegend, Alexa Fluor® 647 Annexin V, Cat #640943
Live/Dead Yellow: LIVE/DEAD™ Fixable Yellow Dead Cell Stain Kit, Cat #L34968
RPMI1640: Hyclone RPMI 1640 medium with HEPES, L-glutamine, Cat #SH30255.FS; RPMI 1640 Medium, no folic acid, Gibco, CAT #27016021
HI-FBS: HyClone Characterized FBS, US Origin, 500 mL, Heat-inactivated,

Cat #SH30071.03HI
CSFE: eBioscience™ CFSE, Cat #65-0850-84
Complete Tumor growth medium: RPMI1640+10% HI-FBS

### Methods:

1.THP-1, KB, Toledo and HL60 Tumor cells grown in Folate acid free RPMI1640 (10% HIFBS) Toledo cells raised in normal RPMI1640 medium were pre-labeled with CFSE (1 uM) and resuspended at 0.5×10⁶ cells/mL in tumor cultural medium
2. GDT cells were prepared and resuspended at 3×10⁶ cells/mL in tumor cultural medium
3. 70 ul T cells were mixed with 140 ul tumor cells to make E:T=3:1 in 96-well U bottom plates
4. 50 ul 3-fold serial diluted GDT cells were mixed 100 ul tumor cells in Triplicate (KB cells were seeded in ultra low binding 96-well plate, while others were in regular tissue culture treated 96-well plate
5. Incubate mixture in tissue cultural incubator overnight
6. Spin cells down at 300 g for 5 min at RT
7. Resuspend cells in each well with 30 ul PBS containing live/dead yellow (1:500), and leave them in dark at RT for 10 min
8. Refill each well with 200 ul FACs buffer (PBS+2% HI-FBS), and spin cells down
9. Discard supernatant and resuspend cells in each well with 30 ul Annexin V antibody (1:100 diluted in Annexin V binding buffer)
10. Stain cells in dark at RT for 15 min
11. Add 90 ul Annexin V buffer
12. Run them at 50 ul out of 120 ul total volume by flowcytometry.

The cytotoxicity assay was conducted using flow cytometry. Effector cells (“E”) included folic acid-conjugated γδT cells, non-conjugated γδT cells, and non-conjugated γδT cells with free folic acid. The target cells (“T”) comprised THP-1, KB, HL-60, and Toledo tumor cells.

To differentiate between effector and target cells, the tumor cells were pre-stained with 1 μM CFSE in DPBS for 10 minutes at room temperature in the dark. Excess CFSE was then removed by washing the cells twice with complete tumor culture medium (folic acid-free RPMI 1640 containing 10% HI-FBS). CFSE-labeled tumor cells were resuspended at a concentration of 0.5×10⁶ cells/ml in complete tumor culture medium, and 100 μl of this suspension was added to each well of a non-tissue culture-treated 96-well plate. Folic acid-conjugated and non-conjugated γδT cells were adjusted to a concentration of 3×10⁶ cells/ml in complete tumor culture medium, and three consecutive 3-fold serial dilutions were performed in a 96-well plate. Subsequently, 50 μl of γδT cells from each dilution was added to the wells containing tumor cells, resulting in E ratios of 3:1, 1:1, 1:3, and 1:9. All experimental and control groups were set up in triplicate. The plates were then transferred to a humidified tissue culture incubator at 37° C. with 5% CO₂.

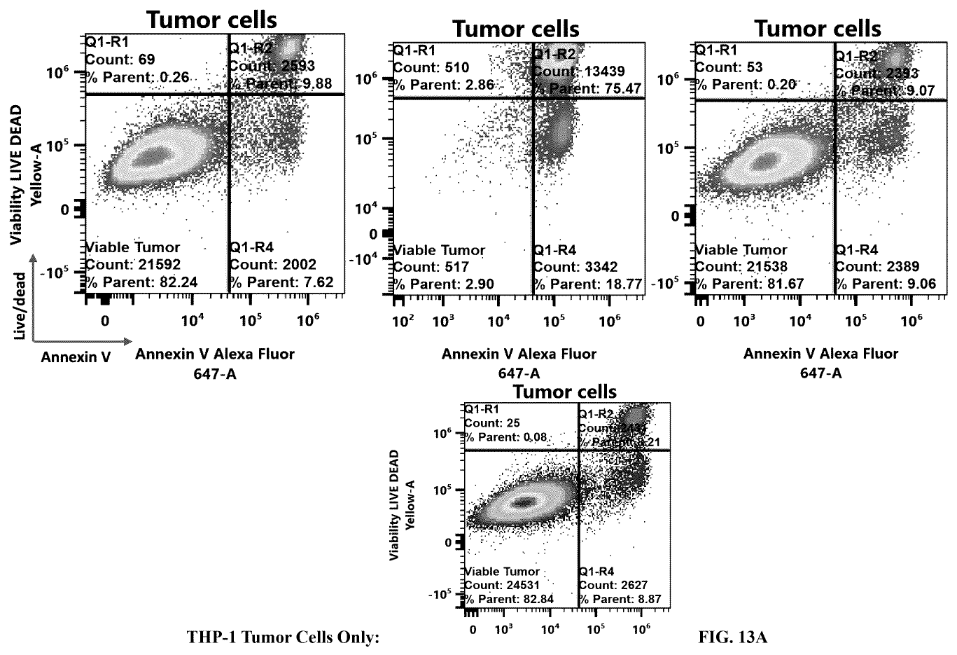

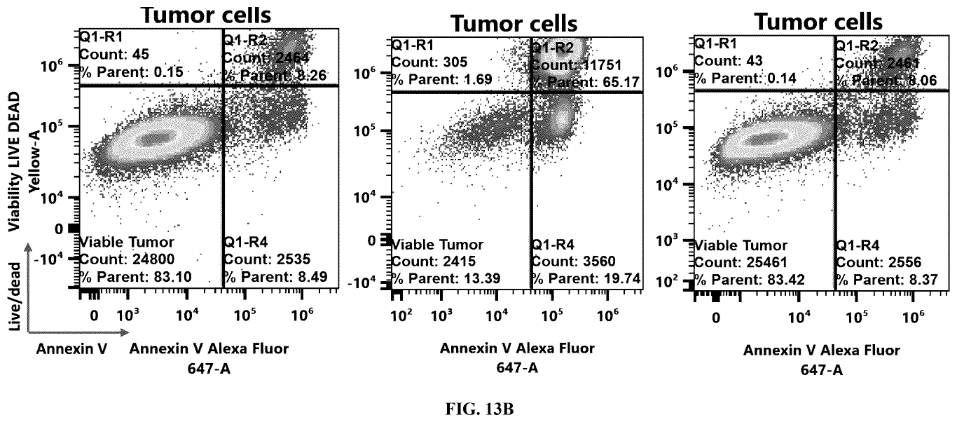

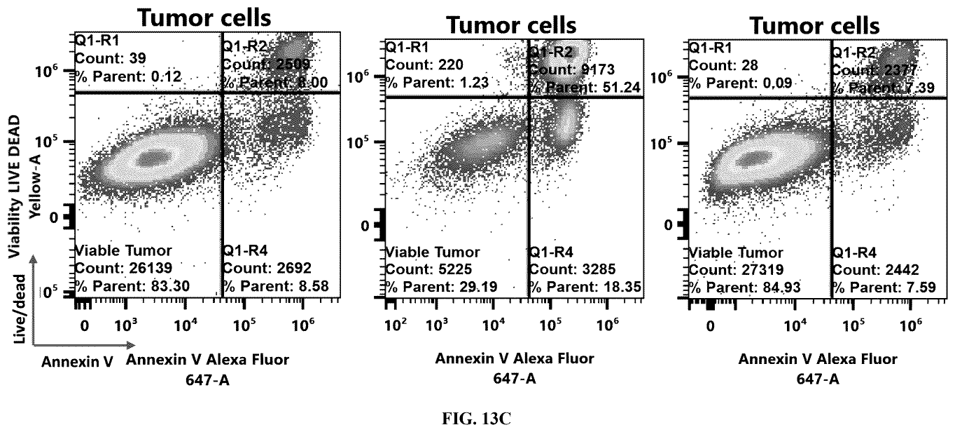

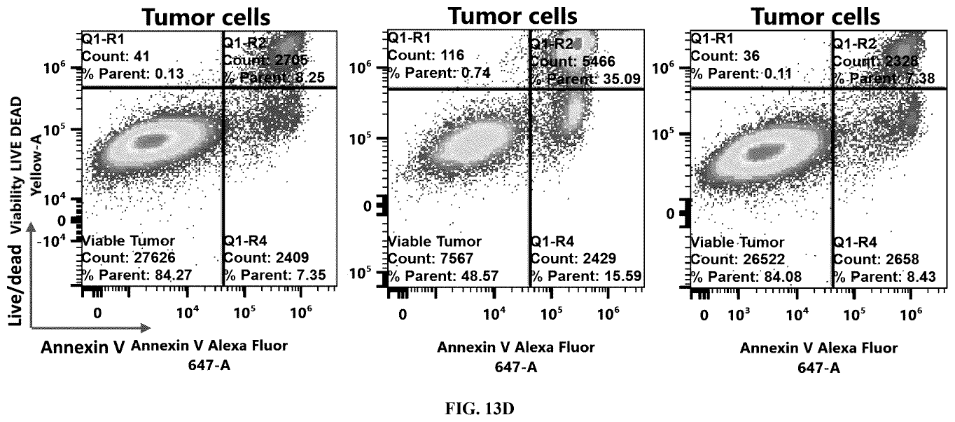

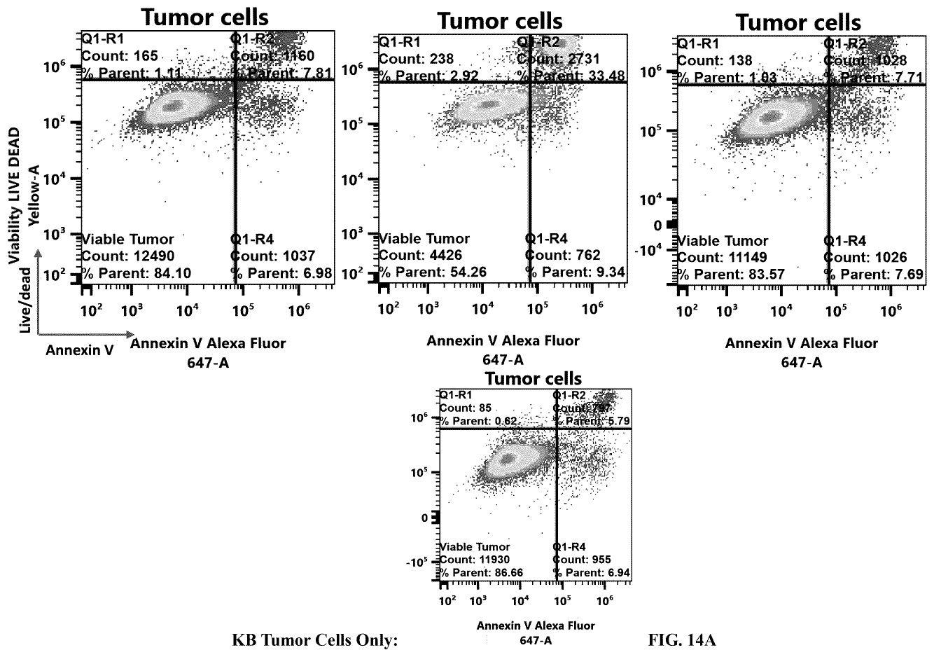

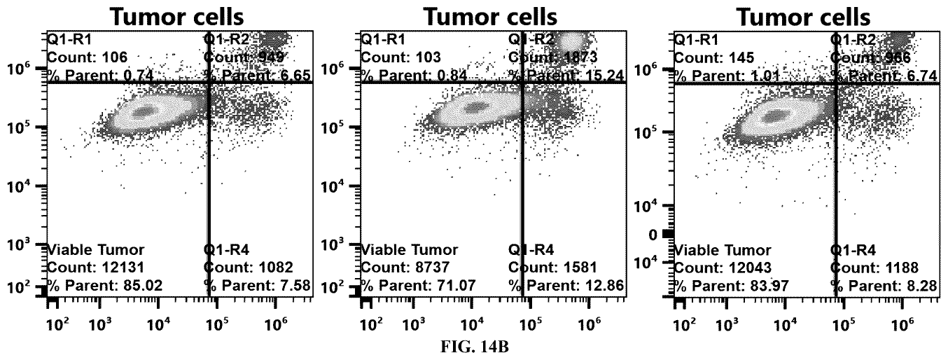

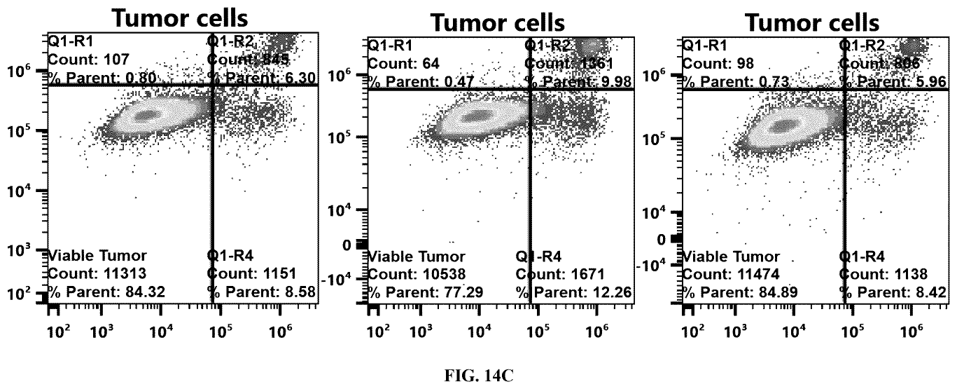

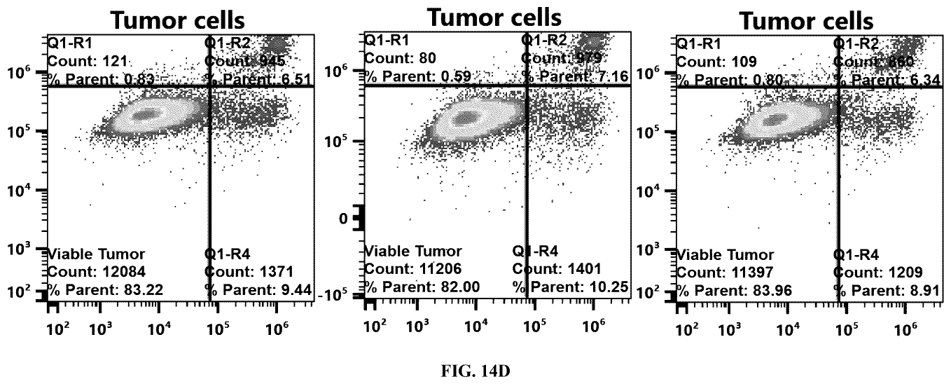

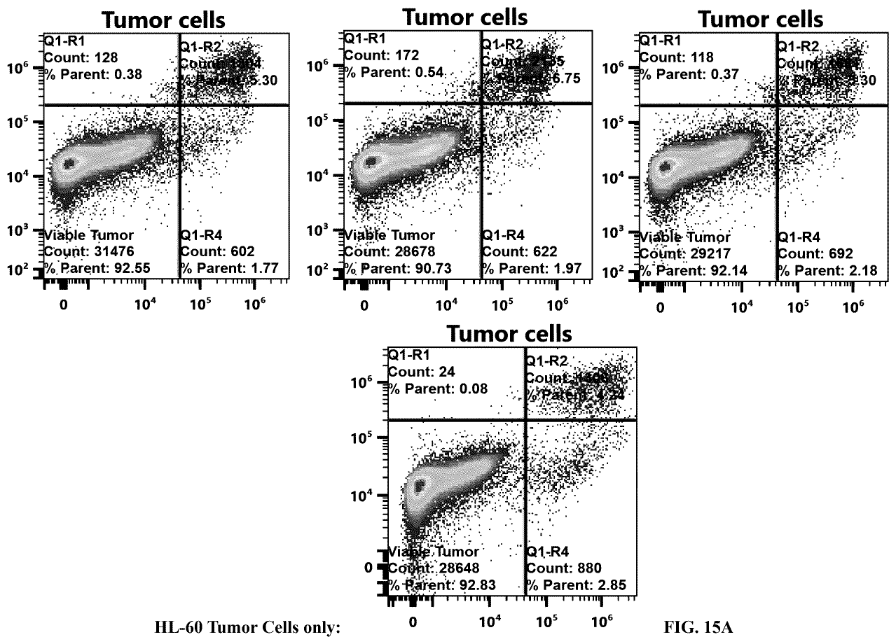

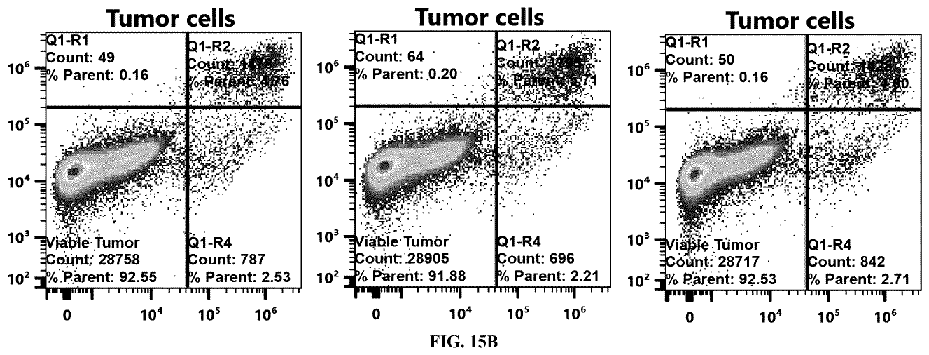

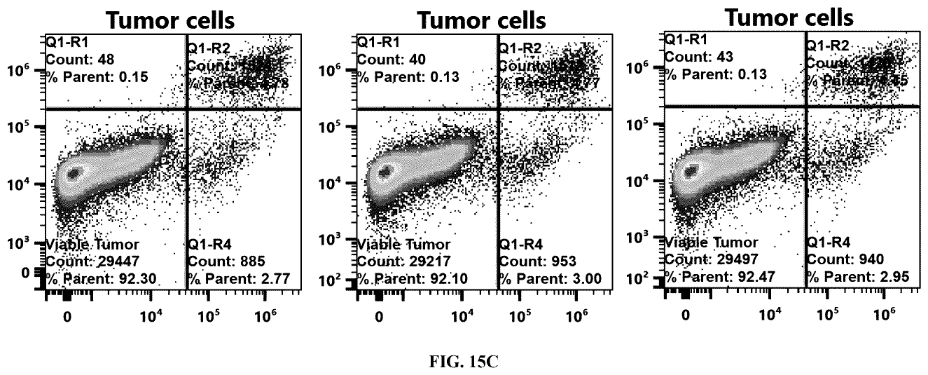

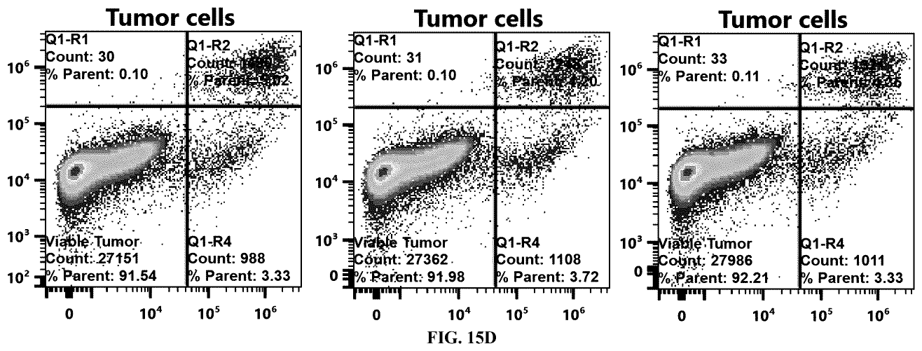

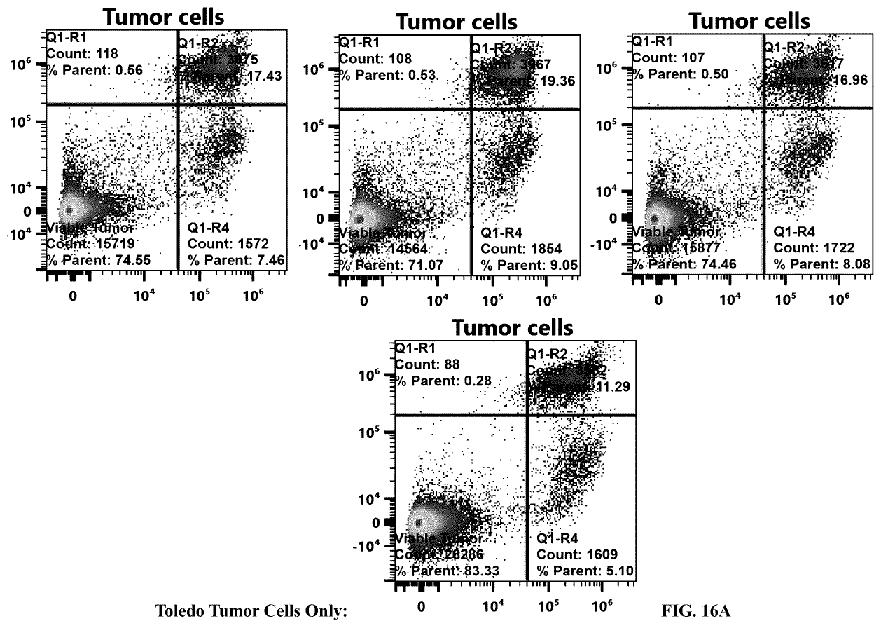

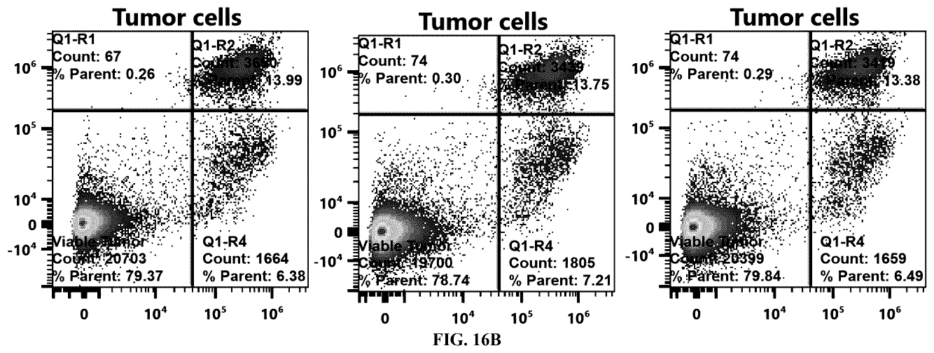

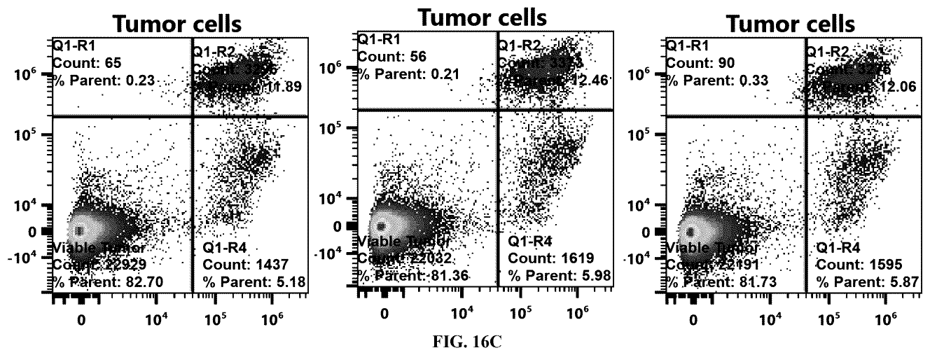

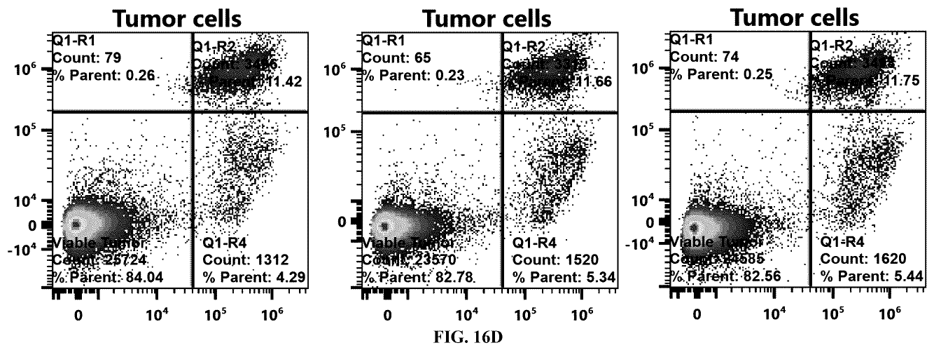

After overnight incubation, the plates were centrifuged at 300×g for 5 minutes at room temperature, and the supernatant was discarded. Cells in each well were stained with 30 μl of DPBS containing live/dead fixable yellow dye (diluted 1:500) for 10 minutes at room temperature in the dark. Following staining, 150 μl of FACs buffer (DPBS with 2% HI-FBS) was added to each well, and the plates were centrifuged again at 300×g for 5 minutes at room temperature. After removing the supernatant, the cells were stained with 30 μl of Annexin V binding buffer mixed with 1:50 diluted anti-Annexin V monoclonal antibody labeled with Alexa Fluor 647 for 20 minutes at room temperature in the dark. After staining, 30 μl of the sample was collected for flow cytometry analysis, with 50 μl of Annexin V binding buffer added to each well (30 μl out of a total 80 μl volume). Viable tumor cells were defined as double-negative for both live/dead yellow and Annexin V staining. The percentage of apoptotic or dead tumor cells was determined based on Annexin V-positive and/or live/dead positive staining. See FIGS. 13-16, for THP-1, KB, HL-60, and Toledo tumor cells, respectively.

### Results

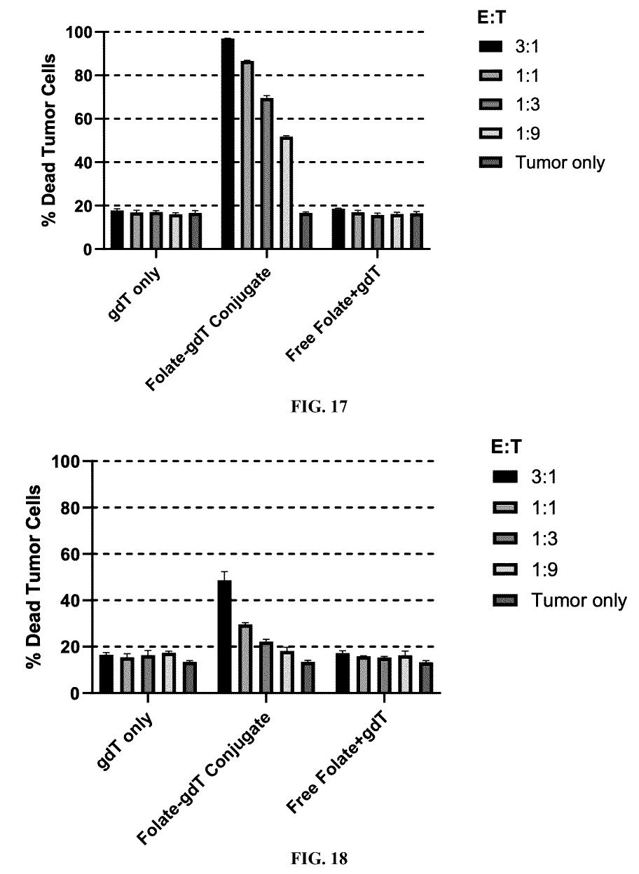

At 3:1E:T ratio, approximately 97% of THP-1 cells were killed by folic acid-conjugated γδT cells. FIG. 17. This efficacy decreased to about 86%, 70%, and 51% as the E:T ratio was reduced to 1:1, 1:3, and 1:9, respectively. In contrast, non-conjugated γδT cells or non-conjugated γδT cells with free folic acid did not exhibit significant cytotoxicity against THP-1 cells, demonstrating that the observed cytotoxicity was dose-dependent and specifically induced by folic acid-conjugated γδT cells. For KB cells, the rate of tumor cells killed is approximately 50%, 30%, 22%, and 18%, compared to about 15% of control (tumor cells only). FIG. 18.

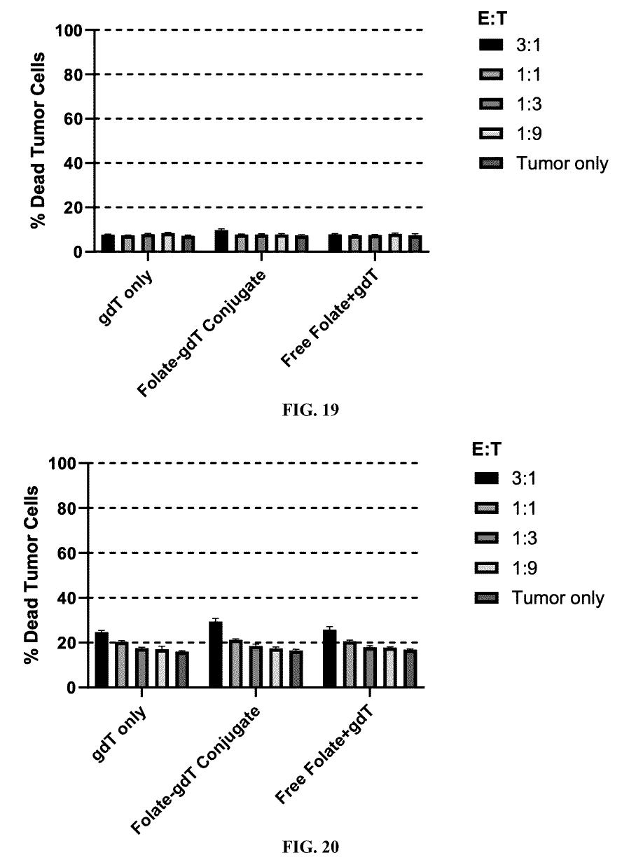

When co-cultured with HL-60 (FIG. 19) or Toledo cells (FIG. 20), which do not express high levels of FOLR1 or FOLR2, neither conjugated nor non-conjugated γδT cells induced significant tumor cell killing.

The observed cytotoxicity is attributable to the targeting of folate receptors by folic acid-conjugated γδT cells.

Example 10. This example evaluates conjugated folic acid on γδT cell surface

To assess the relative amount of folic acid conjugated to the γδT cell surface, immunostaining using flow cytometry was conducted. A total of 100 μL of γδT cells, at a concentration of 2×10⁶ cells/mL, either conjugated with Folic Acid or non-conjugated, were centrifuged at 300×g for 5 minutes at room temperature. The cells were then washed twice with FACS buffer (DPBS with 2% heat-inactivated FBS).

Next, the cells were resuspended in 100 μL of FACS buffer containing anti-Folic Acid mouse monoclonal antibody and incubated at 4° C. in the dark for 1 hour. After two additional washes with 150 μL of FACS buffer, the cells were incubated with 40 μL of FACS buffer containing APC-goat anti-mouse IgG (diluted 1:200) for 20 minutes at room temperature in the dark. Finally, the cells were washed twice with FACS buffer and resuspended in 100 μL of FACS buffer for flow cytometric analysis.

### Results

As shown in FIG. 12, compared with non-conjugated γδT cells, Folic Acid conjugated γδT cells showed significant anti-Folic Acid staining.

Example 11: This example shows results of in vivo efficacy study in THP-1 human leukemia xenograft model.

Fifteen female NSG (code #005557) mice were purchased from the Jackson Laboratory (Bar Harbor, ME). At the time of receipt, the animals were approximately six weeks of age and weighed between 16 g and 20 g. All fifteen animals were utilized in the described efficacy study

Firefly Luciferase expressing THP-1 Cell Line (Human leukemia monocytic cell line, #78409, BPS Bioscience (San Diego, CA)) was cultured per the manufacturer's instructions with specified modifications. Briefly, a cryovial of cells was thawed for about 60 sec in a 37° C. water bath and transferred into 9 mL pre-warmed cell growth media (CGM). The CGM consisted of Folic acid-free RPMI 1640 (#27016021, Gibco) supplemented with 10% fetal bovine serum (FBS) and 1% Penicillin-Streptomycin (PS), Hygromycin B. The cells were centrifugated at 300×g for 5 min, washed once with 5-mL CGM, and seeded into a T-25 flask incubated at 37° C., 5% CO₂. Medium was changed after 72 h to accommodate reduced growth rate. Cultures were passaged twice weekly in T-75 flask at 0.2-2×10⁶ cells/mL, using the same CGM composition.

Cancer Cell Inoculation: After 2 weeks on folic acid-deficient diet and acclimation, animals were intravenously inoculated via tail vein with a single-cell suspension of >90% viable THP-1-Luc tumor cells. Cells were washed twice and resuspended in 0.2 mL serum-free folic acid-deficient RPMI-1640 medium. Prior to inoculation, luciferase expression was confirmed using the Steady-Glo Luciferase Assay System (Promega). Passage numbers at seeding and final use were less than 10.

Fifteen NSG mice were enrolled in the efficacy study. Animals were weighed and assigned to receive an equal inoculum of 4×10⁶ THP-1-Luc cells per mouse via intravenous injection. The day of inoculation was designated as Day 0.

| | Group | # of Mice Per | # of Tumor | | Treatment Dose ROA | Dose |
|---|---|---|---|---|---|---|
| Group | Name | Group | Cells/ROA | Treatment | Frequency | Volume |
| G1 | Vehicle | 5 | 4 × 10⁶ cells/IV | PBS | IV, 2x/week for 2 | 200 μL |
| | Control | | | | weeks | |
| G2 | Control | 5 | 4 × 10⁶ cells/IV | gdt | 1 × 10⁷, IV, 2x/week for | 200 μL |
| | | | | | 2 weeks | |
| G3 | TA-1 | 5 | 4 × 10⁶ cells/IV | Folate-gdt | 1 × 10⁷, IV, 2x/week for | 200 μL |
| | | | | conjugates | 2 weeks | |

IVIS luminescence Imaging was performed on Day 0 (post-inoculation), day 7, day 14, and 2 twice weekly thereafter. Body weights and clinical observations were recorded twice weekly. D-Luciferin (15 mg/ml in PBS) was administered via intraperitoneal injection (200 μL, 150 mg/kg). Ten minutes after the injections, whole body (ventral) were imaged and recorded using the Ami HT imaging system (Spectra Imaging)

Animals in a continuing deteriorating condition, severe distress, or pain were humanely euthanized prior to death, or onset of coma. Euthanasia was performed by CO₂ inhalation followed by cervical dislocation.

Body weight data are presented as mean±SEM for each group at each time point. Statistical comparisons among groups at the final time point were performed using one-way ANOVA followed by multiple comparison tests. Analyses were conducted using GraphPad Prism software. A p-value <0.05 was considered statistically significant.

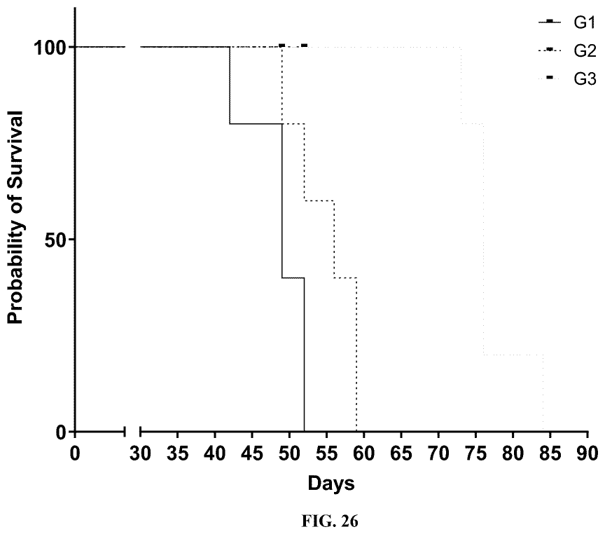

Result: Bioluminescence imaging was used to monitor tumor progression in three groups of NSG mice injected with THP-1-Luc cells: Vehicle control (G1), gdt (G2), and folate-gdt conjugates (G3). Images were acquired at multiple time points from Day 0 Day 84 post-inoculation. Representative IVIS images (FIG. 25) show differential tumor progression by treatment group.

Referring to FIG. 25, in the G1 group, tumor signals progressively increased in intensity and spread over time, demonstrating tumor growth and progression. The G2 treatment group showed a similar pattern of increasing luminescence signal, indicating comparable tumor burden to the control group at all observed time points. In contrast, the G3 treatment group exhibited markedly reduced luminescence signals across all observed time points, suggesting a significant inhibition of tumor growth relative to both the Vehicle Control and G2 groups.

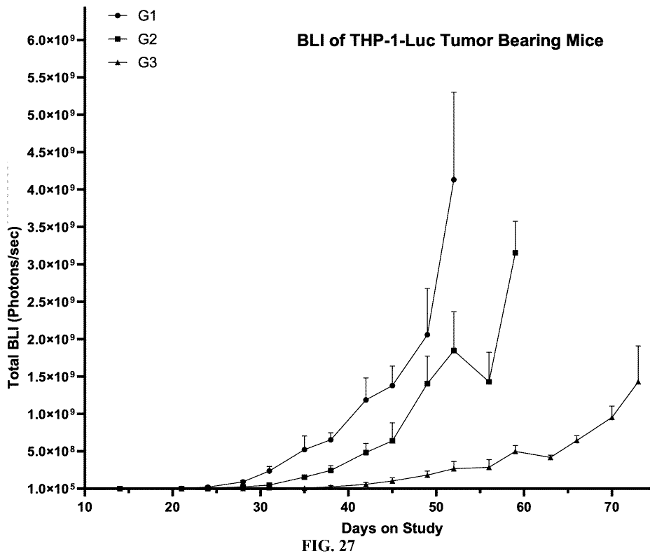

These imaging results provide visual and time-based evidence of the anti-tumor efficacy of the G3 treatment, as reflected by a lower tumor burden and delayed tumor progression compared to the other groups. As shown in FIG. 26, G3 demonstrated a statistically significant improvement in overall survival compared to G1, with a 27-day extension in median survival and a 32-day delay in complete mortality. G2 exhibited minimal therapeutic effect, resulting in only a 7-day extension in median survival and delay in mortality, which was not statistically significant relative to vehicle-treated controls.

The total bioluminescence imaging (BLI) signal was used as a quantitative measure of tumor burden, wherein higher signal intensity corresponds to increased tumor growth. See FIG. 27. In the present study, G1 exhibited rapid tumor progression as reflected by a marked increase in BLI signal. G2 demonstrated a partial inhibitory effect on tumor growth. G3 exhibited the greatest tumor suppression, with only minimal increases in BLI signal over the course of the study.

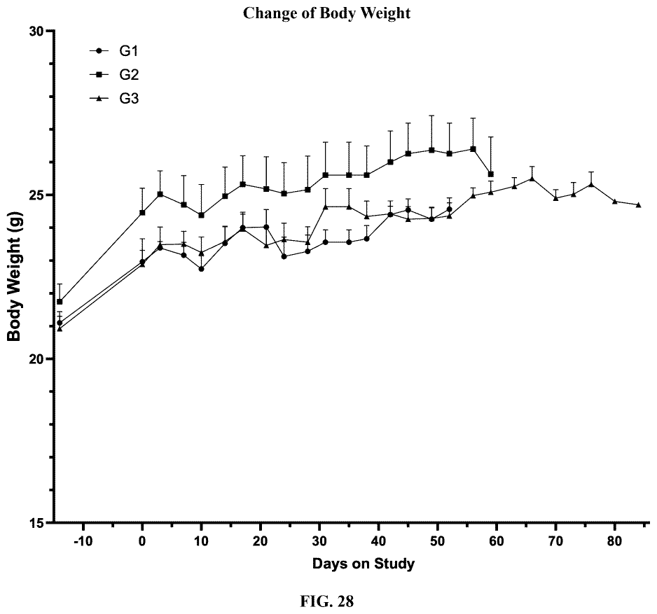

Body weight was monitored as an indicator of general health and treatment tolerability. As shown in FIG. 28, across all treatment groups, body weight remained stable or exhibited a slight increase during the study period. In particular, animals in G3 maintained stable body weight, indicating of a favorable safety profile in conjunction with observed anti-tumor activity. These results demonstrate that G3 exhibits both potent in vivo efficacy and acceptable tolerability in the tested animal model.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A surface modified immune cell comprising (i) a linker that is covalently bound to an immune cell surface and (ii) a ligand covalently bound to said linker, wherein said ligand comprises folate, 5-methyltetrahydrofolate, methotrexate, pralatrexate, raltitrexed, pemetrexed, or a mixture thereof.

2. The surface modified immune cell of claim 1, wherein said immune cell is an innate immune cell.

3. The surface modified immune cell of claim 1, wherein said immune cell is a T cell, B cell, natural killer (NK) cell, macrophage, neutrophil, dendritic cell, mast cell, eosinophil, or basophil.

4. The surface modified immune cell of claim 1, wherein said immune cell is gdT cell.

5. The surface modified immune cell of claim 1, wherein said linker comprises polyethylene glycol (PEG), polyvinyl acid (PVA), polycaprolactone (PCL), polylactic acid (PLA), poly(lactic-co-glycolic acid) (PLGA), polyurethane (PU), polyglycolic acid (PGA), polyethylene oxide (PEO), polypeptide, aliphatic chain, or a combination thereof.

6. The surface modified immune cell of claim 1, wherein said linker comprises from about two to about ten monomeric ethylene glycol units.

7. The surface modified immune cell of claim 1, wherein said ligand comprises racemic mixture of folate, or an enantiomerically enriched folate.

8. The surface modified immune cell of claim 1, wherein said ligand comprises D-folate, L-folate, or a combination thereof.

9. A method for treating cancer in a subject, said method comprising administering a composition comprising a surface modified immune cell of claim 1.

10. The method of claim 9, wherein said cancer is a solid cancer.

11. The method of claim 9, wherein said cancer comprises ovarian cancer, breast cancer, lung cancer, stomach cancer, colorectal cancer, brain cancer, melanoma, sarcoma, lymphoma, blastoma, carcinosarcoma, carcinoma, kidney cancer, bladder cancer, pancreatic cancer, cervical cancer, or uterine cancer.

12. The method of claim 9, wherein said cancer is a blood cancer.

13. The method of claim 9, where said cancer comprises multiple myeloma or myelogenous leukemias.

14. The method of claim 9, wherein said immune cell is an innate immune cell.

15. The method of claim 9, wherein said immune cell is an allogeneic immune cell.

16. The method of claim 9, wherein said composition comprises a plurality of allogeneic immune cells.

17. A method for producing a surface modified immune cell of claim 1, wherein said method comprising:
(i)
(a) contacting an immune cell with a linker having a proximal end and a distal end under conditions sufficient to form a covalent bond between said immune cell surface and said proximal end of said linker; and
(b) contacting said immune cell of step (a) with a ligand under conditions sufficient to form a covalent bond between said distal end of said linker and said ligand to produce said surface modified immune cell, wherein said ligand comprises folate, 5-methyltetrahydrofolate, methotrexate, pralatrexate, raltitrexed, pemetrexed, or a mixture thereof; or
(ii) contacting an immune cell with a linker having a proximal end and a distal end under conditions sufficient to form a covalent bond between said immune cell surface and said proximal end of said linker to produce said surface modified immune cell, wherein said distal end of said linker is covalently bonded to a ligand, wherein said ligand comprises folate, 5-methyltetrahydrofolate, methotrexate, pralatrexate, raltitrexed, pemetrexed, or a mixture thereof.

18. The method of claim 17, wherein said linker comprises polyethylene glycol (PEG), polyvinyl acid (PVA), polycaprolactone (PCL), polylactic acid (PLA), poly(lactic-co-glycolic acid) (PLGA), polyurethane (PU), polyglycolic acid (PGA), polyethylene oxide (PEO), polypeptide, aliphatic chain, or a combination thereof.

19. The method of claim 17, wherein said immune cell is allogeneic immune cell relative to said target cell.

20. The method of claim 17, wherein said immune cell is a T cell, B cell, natural killer (NK) cell, macrophage, neutrophil, dendritic cell, mast cell, eosinophil, or basophil.
